(19) United States
(12) Patent Application Publication
(10) Pub. No.: US 2023/0082580 A1
(43) Pub. Date: Mar. 16, 2023

(54) BODY-WORN WIRELESS TWO-WAY COMMUNICATION SYSTEM AND METHOD OF USE

(71) Applicant: Integrated Tactical Technologies, LLC, Southern Pines, NC (US)

(72) Inventors: Michael PARÉ, San Carlos, CA (US); Timothy L. PROULX, Santa Cruz, CA (US); Peter HADROVIC, Southern Pines, NC (US)

(73) Assignee: Integrated Tactical Technologies, LLC, Southern Pines, NC (US)

(21) Appl. No.: 17/929,621

(22) Filed: Sep. 2, 2022

Related U.S. Application Data
(63) Continuation of application No. PCT/US2021/021198, filed on Mar. 5, 2021.
(60) Provisional application No. 62/985,793, filed on Mar. 5, 2020.

(51) Int. Cl. H04B 13/00; H04B 5/00

(52) U.S. Cl. CPC H04B 13/005; H04B 5/0093

(57) ABSTRACT

The body-worn wireless two-way communication system comprises a non-invasive and non-implanted system which allows for clear wireless two-way communications. This system is generally comprised of a mouthpiece component, relay component, infrastructure communication device, an optional earpiece component, and an optional system control which may interface with the relay component.

### CROSS-REFERENCE TO RELATED APPLICATIONS

This application is a continuation of International App. No. PCT/US2021/021198 filed Mar. 5, 2021, which claims the benefit of priority to U.S. Prov. 62/985,793 filed Mar. 5, 2020, which is incorporated herein by reference in its entirety.

### FIELD OF THE INVENTION

Systems, devices, and methods for two-way communication are disclosed. More specifically, systems, devices, and methods for body-worn wireless one-way or two-way communication are disclosed that have a mouthpiece component and an optional earpiece component. The mouthpiece and/or earpiece component can each provide one-way or two-way communication.

### BACKGROUND OF THE INVENTION

Covert operations necessitate communication systems with very specific and stringent requirements. Such operations typically require clear two-way communications systems which are not visible or are minimally visible to an outside observer, that allow for complete audio awareness of the environment, and which can be utilized in a less than overt manner.

Current technology has not been able to completely meet these requirements as existing systems invariably include some small visible component observable to a perceptive onlooker. Current communication systems also suffer from an inability to effectively communicate the user's speech when in very loud environments due to poor signal to noise ratio.

The warfighter/operator requires clear two-way communications in extreme operating conditions, and in a form factor that allows discreet use and that can be adaptively integrated and used with other equipment and protective gear and do so with minimal restriction of movement or potential for entanglement.

Accordingly, there is a need for a body-worn wireless communication system with intra-oral and/or in-ear transceiver that eliminates the wires currently associated with radio communication systems, allows for more mobility, reduces weight, eliminates the visual signature of communications gear, and improves overall communication capability in all operating conditions, especially extreme conditions that prevent or severely impede audio communication.

### BRIEF SUMMARY OF THE INVENTION

The present disclosure relates generally to two-way communication with a mouthpiece component and/or an earpiece component. More specifically, the present disclosure relates to one-way or two-way communication with a mouthpiece component and an optional earpiece component using various wireless coupling methods such as magnetic coupling, NFMI, magnetic resonance, RF, or wireless body area networking (WBAN) including magnetic human body communication (mHBC), such as magnetic resonance coupling.

The communication system disclosed allows the operator or user to hear their environment by leaving their hearing unobstructed and thereby enables complete audio situational awareness by the operator.

Generally, one variation of a two-way communication system may comprise a mouthpiece component removably engageable within a mouth of a user, a relay component in wireless communication with the mouthpiece component via a wireless body area networking signal or a low-frequency non-propagating inductive field, and an infrastructure communication device configured to transmit wireless signals to and receive wireless signals from the relay component, wherein the relay component is configured to interface between the mouthpiece component and the infrastructure communication device.

A further variation may comprise a mouthpiece component removably engageable within a mouth of a user, a first earpiece component positionable with or in proximity to an ear of the user, a relay component in wireless communication with the mouthpiece component and/or first earpiece component via a wireless body area networking signal or a low-frequency non-propagating inductive field, and an infrastructure communication device configured to transmit wireless signals to or receive wireless signals from the relay component, wherein the relay component is configured to interface between the mouthpiece component and/or the first ear piece component and the infrastructure communication device.

In one variation of a method of providing two-way communication, the method may generally include receiving a signal via an infrastructure communication device carried by a user and configured to transmit wireless signals to or receive wireless signals from a relay component via a wireless body area networking signal or a low-frequency non-propagating inductive field, transmitting the signal to the relay component which is configured to interface between a mouthpiece component and the infrastructure communication device, further transmitting the signal to the mouthpiece component which is configured for temporary securement upon a tooth or teeth of the user, wherein the relay component is in wireless communication with the mouthpiece component via a wireless body area networking signal or a low-frequency non-propagating inductive field, and vibrationally conducting the signal through the mouth of the user via the mouthpiece component.

### BRIEF DESCRIPTION OF THE DRAWINGS

The drawings shown and described are exemplary embodiments and non-limiting. Like reference numerals indicate identical or functionally equivalent features throughout.

FIG. 1 shows a schematic representation of a variation of a two-way communication system having a mouthpiece component, a relay component, an infrastructure communication device, and/or a system control.

FIG. 2A shows a variation of a mouthpiece component having an integrated NFMI antenna/coil.

FIG. 2B shows a partial cross-sectional illustration of a variation of a MEMS-type microphone assembly securable within a user's mouth.

FIG. 2C shows a perspective assembly view of a variation of the mouthpiece component of FIG. 1 that is securable within the mouth and upon one or more teeth of a user.

FIG. 3A show a schematic illustration of a variation of the two-way communication system of FIG. 1 where the mouthpiece component communicates with the infrastructure communications device via the relay component.

FIG. 3B shows a schematic illustration of another variation of the two-way communication system of FIG. 3A, where the relay component is mounted on the infrastructure communication device.

FIG. 3C shows a schematic illustration of a variation of the two-way communication system, where the relay component is configured to be mounted on a shoulder.

FIG. 3D shows a schematic illustration of a variation of the two-way communication system, where the relay component is communicable with the mouthpiece component and optional earpiece components.

FIG. 3E shows a schematic illustration of a variation of the two-way communication system, where the relay component mounted on the infrastructure communication device is communicable with the mouthpiece component and optional earpiece components.

FIG. 4 shows a schematic representation of a variation of the two-way communication system of FIG. 1 with the earpiece component.

FIG. 5 shows a schematic representation of a variation of the two-way communication system of FIG. 4 having audio/data links utilizing body conduction.

FIG. 6 shows a schematic representation of a variation of the earpiece component of FIG. 4.

FIG. 7 shows a schematic illustration of a variation of various audio modes of the earpiece component.

FIG. 8 shows a schematic illustration of a variation of various communication modes of the two-way communication system of FIG. 4 for incoming communication.

### DETAILED DESCRIPTION OF THE INVENTION

Systems, devices and methods for two-way communication are disclosed that can provide reliable and/or clear incoming and/or outgoing communication. The systems, devices, and methods disclosed can provide reliable and/or clear communication in any environment, for example, low noise environments, medium noise environments, high noise environments, or any combination thereof. The systems, devices and methods disclosed can be adjusted in real-time, manually or automatically, to advantageously function in one or multiple environments that have, for example, dynamic noise conditions.

The systems, devices and methods disclosed can be partially or completely visible or invisible to an outside observer when in use by an operator or user. For example, one or multiple components of the systems, devices and methods disclosed— including the entire system—can be partially or completely visible or invisible, for example, to another person, an image capturing system (e.g., a camera- or video-based surveillance system), or any combination thereof.

The communication systems disclosed can be non-invasive systems, invasive systems, non-implanted systems, implanted systems, or any combination thereof. For example, the communication systems disclosed can be non-invasive and non-implanted systems.

The communication systems disclosed can allow for clear two-way half-duplex and/or full-duplex communications.

The systems disclosed can allow the operator or user to hear their environment by leaving their hearing unobstructed and thereby enable complete audio situational awareness by the operator. With the addition of hearing protection, the same systems instantly convert into complete tactical and general purpose communication systems, functional in high noise environments. The hearing protection may comprise pass-through hearing protection still allowing for full situational awareness.

One aspect of the systems disclosed is the use of an in-mouth communication device (also referred to as a mouthpiece component) utilizing bone conduction driven into the teeth for received audio, and an integrated microphone as part of the mouthpiece component. For example, the mouthpiece component can have one or multiple mouthpiece microphones. The mouthpiece microphone(s) can be attached to or integrated with the mouthpiece component. Using the in-mouth communication device, the operator may leave the ears completely open and unobstructed to enable the user to be situationally aware. Clear communications are possible while eliminating all cables (e.g., to the head) and headsets.

The communication systems disclosed are particularly well suited for low-visibility applications as all parts of the system may either be hidden under clothes or are otherwise not visible to an external observer. For loud environments where in-ear or on-ear hearing protection is warranted or otherwise desirable, the same communication systems may be used as a general purpose and/or tactical communication system by incorporating hearing protection with pass-through hearing protection allowing situational awareness to the user. This not only provides safety for the user, but also allows for reduced external noise competition for the user's attention, such that incoming audio communications may be heard and understood with more clarity. Furthermore, having the microphone embedded in the mouthpiece component enables extremely good noise shielding capabilities such that clear outgoing audio transmissions are possible even in loud environments (e.g., deafeningly loud environments). In cases where hearing protection is acceptable, the system may also be used as a general purpose communication system.

One variation for the communications system is for a fully wireless body-worn communications system suitable for extreme operating conditions or for discreet use that can be implemented using several wireless transmission paths, including, e.g., over-air using traditional RF, near field magnetic induction (NFMI), or through tissue by utilizing the conductive properties of the body.

The system provides a fully wireless solution by eliminating any wired connection between the relay component and the infrastructure component. This provides several key advantages to the user, such as improved mobility, lower weight, lower visibility to the observer, improved ease of use, and reduced risk of entanglement and injury, etc.

Wireless Body Area Networking (WBAN) technology has been developed and standardized to allow devices to operate on or inside the human body, supporting many medical and consumer applications. As described in the IEEE 802.15.6 standard, WBAN can include several physical layers, including Narrow Band (NB), Ultra-Wideband (UWB) and Human Body Communications (HBC). An alternative method to WBAN radio frequency technology for the body-worn wireless communication system is magnetic induction, including near field magnetic induction (NFMI), which uses a low-frequency non-propagating inductive field to achieve a local wireless bubble around the user, and magnetic HBC (mHBC), in which transmitter and receiver are inductively coupled through the tissue. Various options of interest are summarized in Tables 1 and 2.

TABLE 1
WBAN Physical Layers and Frequency Bands of Interest

| Physical Layer | Type | Frequency Band | |
|---|---|---|---|
| NB | MICS | 402-405 | MHz |
| | ISM | 433-434.8 | MHz |
| | | 902-928 | MHz |
| | | 2.4-2.5 | GHz |
| UWB | Low | 3.1-4.8 | GHz |
| | High | 6.3-7.9 | GHz |
| HBC (eHBC) | Galvanic | 5-50 | MHz |
| | Capacitive | 5-50 | MHz |

TABLE 2
Wireless Magnetic Communications Options

| Type | Frequency Band |
|---|---|
| Over-air NFMI | <15 MHz |
| HBC (mHBC) | <15 MHz |

NB can operate in various frequency bands including Medical Implant Communication Service (“MICS”) (402-405 MHz) through ISM bands (e.g., with frequency bands of 433-434 MHz, 902-928 MHz, and/or 2.4-2.5 GHz) and can support data rates above 60 kbps. (Kwak et al, “An Overview of IEEE 802.15.6 Standard”, Proceedings of 2010 International Symposium of Applied Sciences in Biomedical and Communication Technologies ISABEL, 2010.) Low power transceivers that can support the MICS and 433 MHz ISM bands include the ZL70103 from Microsemi (Aliso Viejo, Calif.), which can be used for medical implant telemetry. The 2.4 GHz band can be supported by chipsets from numerous manufacturers using 802.11 protocols (e.g. Bluetooth, BLE, Zigbee) or custom data transfer protocols. (Thotahewa et al, Ultra-Wideband Wireless Body Area Networks, Springer Science & Business, 2014.) All bands in Table 1 can support WBAN applications, but the MICS and 433 MHz ISM bands would be more appropriate for the over-air link, such as from the mouthpiece component 10 to the relay component 12, due to the limited absorption by tissue and water at these lower frequencies.

UWB can operate in the 3.1-4.8 GHz and 6.3-7.9 GHz bands and can provide several advantages over NB, including higher data rates up to 100 Mbps and very low detectability/probability of interception due to low transmit power and localized fields (only a few feet from a user). Since UWB is outside the congested ISM bands, it also has less likelihood of interference with other wireless systems. However, UWB suffers from the body-shadowing effect due to tissue absorption, so its use on the body is limited to applications where tissue or water does not block the path between wireless components.

Electric field HBC (eHBC) can use the body as the transmission medium for electrical signal transfer using electrodes that are capacitively or galvanically coupled to the body and operates in the 5-50 MHz frequency band per the IEEE 802.15.6 standard. (Zhao et al, “A Review on Human Body Communication: Signal Propagation Model, Communication Performance, and Experimental Issues”, Wireless Communication and Mobile Computing, Wiley/Hindawi, 2017.) The benefits of this technology include low signal attenuation/low power due to the efficiency of signal propagation in the tissue, low signal leakage/detectability since the signal is confined to the tissue and does not radiate, simplified transceiver design due to low operating frequencies, and higher miniaturization since antennas are not required. Capacitive coupling generally propagates farther through the tissue and supports higher data rates due to its higher amplitude, but may be more prone to external interference and other effects such as body movement. Both techniques would support distances from the head to torso and the 50-100 kbps range for audio communication.

In one embodiment, the mouthpiece component 10 can be in direct contact with the mucous membrane of the user's mouth and the earpiece component 100 can be in contact with the soft tissue of the user's ear canal, which would naturally support galvanic electrode coupling. For galvanic coupling, special electrodes in intimate contact with the skin (such as those used for ECGs) can be used for other body worn devices (the relay component or a separate wireless remote control). Each of the transmitting and receiving components can incorporate two or more electrodes which are separated as far as possible to maximize the voltage potential between them. For example, 0.38 mm electrodes spaced at 2.5 mm have been used to achieve detection of transmitted signals, but order of magnitude higher separation is possible with form factors considered here. (Lindsey DP, McKee EL, Hull ML, Howell SM. A new technique for transmission of signals from implantable transducers. IEEE Trans Biomed. Eng. 1998; 54(5):614-619.) Alternatively, capacitive coupling could be achieved by attaching signal electrodes to the skin or mucous membrane of the user's mouth and leaving ground electrodes floating.

As mentioned above, an alternative method to WBAN radio frequency technology for the body-worn wireless communication system is near field magnetic induction (NFMI), which uses a low-frequency (<15 MHz) non-propagating inductive field to achieve a local wireless bubble around the user. A transmit coil or loop antenna can be used to generate a local magnetic field that is picked up by other receiving coil or loop antennas and converted to electrical current. Like eHBC and UWB, NFMI benefits from low power requirements, low detectability, and reduced interference with other wireless devices or signals. It is particularly suitable for operation around/in the body or water since the magnetic field is not absorbed or affected by the medium.

Another form of communication using magnetic fields that does not rely on inductive coupling between two nearby coil/loop antennas uses magnetic fields from a transmitting coil/antenna/transceiver that can be resonantly coupled to a distant receiving coil/antenna/transceiver via propagation through the tissue. This approach has been termed as magnetic human body communication (mHBC) to differentiate from the electrical HBC technology (eHBC) described above. (Park et al, “Magnetic human body communication”, Conf Proc IEEE Eng Med Biol Soc 2015: 1841-4.) As with eHBC, magnetic HBC offers a low power, low detectability option for the body-worn wireless communication system. However, unlike eHBC links, which do not necessarily travel well through many biological tissues, the mHBC link easily travels through tissue, offering significantly reduced path loss and, as a result, reduced transceiver power consumption. To maximize communication distance and minimize path loss, mHBC utilizes resonant coupling (as opposed to inductive coupling), similar to that of resonant wireless power transfer systems.

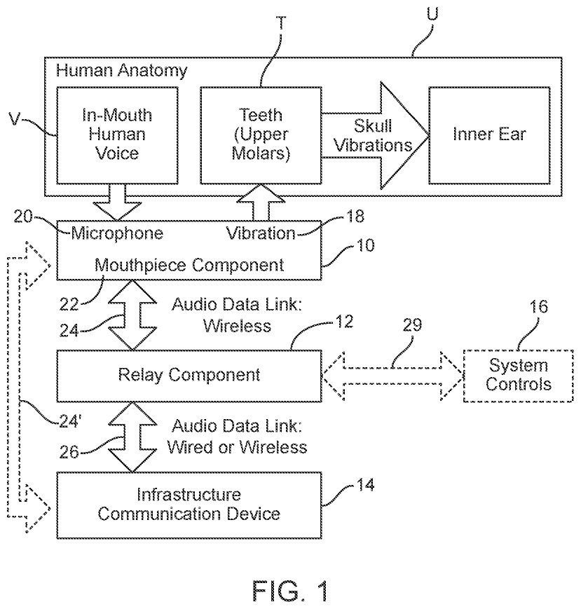

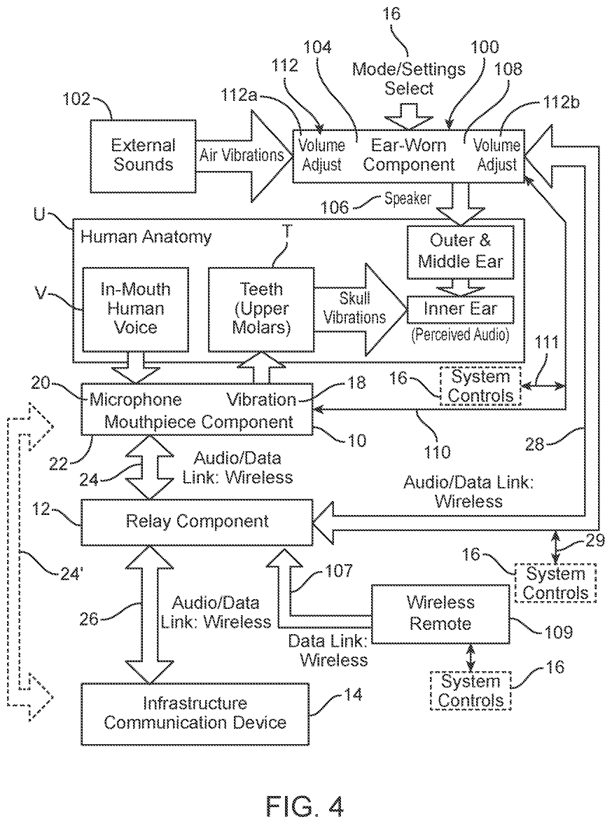

FIG. 1 illustrates that the system can have a mouthpiece component 10, a relay component 12, an infrastructure communication device 14, system controls 16, or any combination thereof. For example, the system can have the mouthpiece component 10, the relay component 12, and the infrastructure communication device 14. As another example, the system can have the mouthpiece component 10, the relay component 12, the infrastructure communication device 14, and the system controls 16. The system controls 16 may interface with the relay component 12, as shown in the schematic diagram of FIG. 1. The system can also include an optional earpiece component(s) 100 (see e.g., FIG. 4, as described in further detail below).

Variations of individual system components are detailed below, including different options for embodiments and particular design attributes.

The mouthpiece component 10 can removably attach or clamp onto or around the molars, e.g., upper back molars, of the operator or user U and allow for normal speech, eating, and drinking without impediment. Additionally, the mouthpiece component 10 may be temporarily secured to the user's tooth or teeth without having to alter the user's dentition in any way such that the user may simply remove the component 10 from the tooth or teeth when finished, leaving the dentition unaltered. When the mouthpiece component 10 is to be used again, it may be simply reinserted within the mouth and secured to the tooth or teeth again. FIG. 1 further illustrates that the mouthpiece component 10 can also utilize bone-conduction technology by actuating one or more transducers 18 incorporated into the mouthpiece component 10 and vibrationally coupled to the teeth T to convey incoming audio to the user, as indicated by the vibrational conductance. The mouthpiece component 10 can also integrate a microphone assembly 20 to capture the user's speech V for outgoing audio transmissions. The mouthpiece component 10 may also incorporate a transmitter and/or receiver such as a transceiver 22 which enables wireless communication 24 with the relay component 12 to receive and transmit audio data.

The wireless link 24 between the mouthpiece 10 and relay component 12 can be utilized in order to get audio data into and out of the mouth area and may utilize any number of wireless data transmission protocols, e.g., near field magnetic induction (NFMI), radio frequency (RF) link such as BLUETOOTH®, NB (MICS or ISM 433M Hz bands), body conduction, and/or acoustic signals at or above human hearing. Alternatively in other variations, a wireless link 24′ (or direct attachment) may be formed directly between the mouthpiece 10 and infrastructure communication device 14, as shown here and further below. Utilizing NFMI is generally preferable to RF due to reduced attenuation through body tissue and also due to a reduced electromagnetic far field profile. Regardless of wireless link implementation, the data can be encrypted to ensure security using, e.g., AES-256 or other encryption standards.

As further illustrated in FIG. 1, if the relay component 12 is a device physically separate from the infrastructure communication device 14, then a wireless link 26 may be established between the relay component 12 and infrastructure communication device 14, which can utilize NFMI or any WBAN wireless link since the connection is outside the body. The system controls 16 can communicate to the relay component 12 via a wireless data link 29 and can also utilize either NFMI or with any of the WBAN options as described previously.

The infrastructure communication device may include devices such as a cell phone, radio, intercom device, etc. NFMI links can advantageously allow for under water usage. Furthermore, if NFMI is used at a fixed frequency, the magnetic field strength may be reduced in order to avoid user-to-user interference when multiple communication systems are worn in close proximity. Otherwise, time-sharing the bandwidth or utilizing dedicated frequency channels may be handled. Where an NFMI link is used with both the relay component 12 and the infrastructure communication device 14, the relay component and infrastructure communication device NFMI links can each operate at a different frequency from one another, or the NFMI link for each can be time-shared/coordinated with each other such that a different frequency is not needed.

Additionally and/or optionally, the relay component 12 may also be implemented such that there are links to two or more separate infrastructure communication devices worn by the same user simultaneously (such as two radios used in tactical operations, to two different teams). Incoming audio is typically designated in current dual-communication systems by driving audio to either the right or left ear in a headset. For a system with a single mouthpiece component 10, a pre-audio notification may be used to alert the user as to which device is the source of incoming audio. If the mouthpiece component 10 includes teeth-drive contact points on both the right and left sides of the mouth, audio may be phased to these drive points such that the perceived audio is coming from the right or left side.

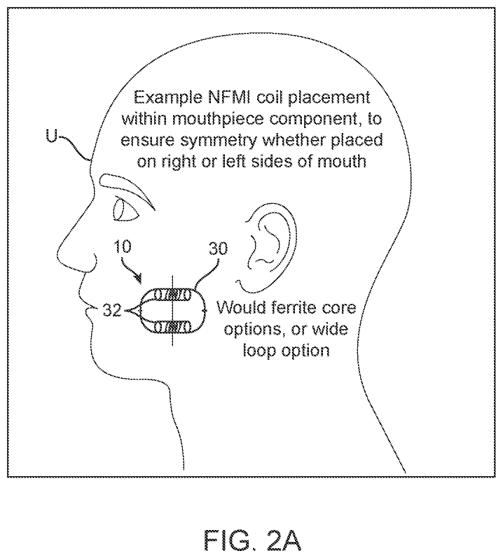

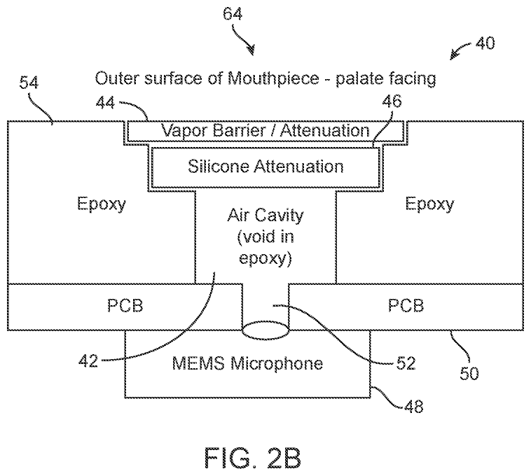

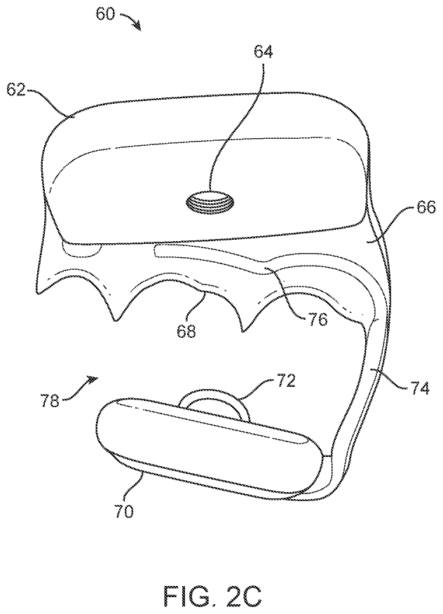

FIG. 2A illustrates that the mouthpiece component 10 can have an antenna 32 (also referred to as a sensing mechanism 32) to receive signals/data (e.g., from the relay component 12). The sensing mechanism 32 can be attached to or integrated with the mouthpiece component 10. The sensing mechanism 32 can desirably allow the mouthpiece component 10 to receive data without a wire and can be, for example, a transducer, electrode, or other sensing mechanism that can function as an antenna. For example, the mouthpiece component 10 can receive data via body conducted signals (such that the sensing mechanism can be an electrode), via acoustic signals sent at or above human hearing to a piezo transducer, via ultrasound, via electromagnetically with an RF antenna, and/or via a magnetic field with a loop/coil antenna. For example, using NFMI for the wireless link means the mouthpiece 10 is utilizing an integrated NFMI antenna/coil 32, as shown in FIG. 2A, which may be completed through either a wire-wound ferrite core antenna or a wide loop option such as a larger loop coil 30. Regardless of implementation, one design consideration is the ability of the mouthpiece component 10 to work similarly whether placed on the right or left upper molars, as different users may require different sides depending on dental anatomy. This means NFMI coil/antenna 32 placement preference is somewhere symmetric, such as along the bottom or top edge of the mouthpiece component 10, e.g., in a vertical or loop configuration.

The mouthpiece component 10 may be comprised of a low-profile housing configured for temporary securement within the mouth and upon the tooth or teeth of the operator. This mouthpiece component 10 utilized may be described in further detail in any one of the following patent references: U.S. Pat. Nos. 7,664,277; 7,682,303; 7,724,911; 7,796,769; 7,801,319; 7,844,064; 7,844,070; 7,854,698; 7,876,906; 7,945,068; 7,974,845; 8,023,676; 8,150,075; 8,160,279; 8,170,242; 8,177,705; 8,224,013; 8,233,654; 8,254,611; 8,270,637; 8,270,638; 8,291,912; 8,295,506; 8,333,203; 8,358,792; 8,433,080; 8,433,082; 8,433,083; 8,503,930; 8,577,066; 8,585,575; 8,588,447; 8,649,535; 8,649,536; 8,649,543; 8,660,278; 8,712,077; 8,712,078; 8,795,172; 8,867,994; 9,113,262; 9,143,873; 9,185,485; 9,247,332 and U.S. patent application Ser. No. 16/129,536 filed Sep. 12, 2018. These patent references are incorporated herein by reference in their entirety and for any purpose herein.

FIG. 2B illustrates that the integrated microphone 20 capability in the mouthpiece component 10 may be implemented with either, e.g., a silicon MEMS-type, piezo MEMS, electret or magnetic air microphone, a PVDF film type vibrational sensor, as described in further detail below.

For an air-type (e.g. MEMS) microphone assembly 40, as shown in the partial cross-sectional illustration of FIG. 2B, the in-mouth sound pressure levels are high (e.g., up to ˜150 dBSPL) necessitating audio attenuation as part of the microphone assembly to prevent clipping and distortion. Air type microphones also typically utilize the presence of a small air cavity 42, so this implementation may include a waterproof barrier 44 such as a tape or film as well as an attenuation element 46 such as a silicone disk, as part of the audio path to the air cavity 42 created for the microphone 48. The air cavity 42 may be formed in part by a first substrate 50, e.g., PCB defining an opening or channel 52 to the microphone 48, and a second substrate 54, e.g., housing structure formed from a material such as epoxy, which may form the sidewalls of the air cavity 42. The attenuation element 46 may be mounted upon the second substrate 54 along with the barrier 44 positioned upon the attenuation element 46 and the second substrate 54, as shown. The portion of the assembly 40 upon which the barrier 44 is positioned may be positioned adjacent to the outer surface of the mouthpiece component 10, e.g., facing the palate of the user. In other variations, the microphone 48 may be positioned on the opposite surface of the substrate 50, e.g., on the same side of the incoming audio signals, such that the opening or channel 52 through the substrate 50 may be omitted entirely.

In order to handle the limited dynamic range of typical conventional air-type microphones, the attenuation 46 added as part of the assembly 40 may be typically selected to still allow normal as well as low-voice speech levels, but enough attenuation such that loud talking or yelling will not be distorted, e.g., attenuation on the order of −30 dB is sufficient to prevent audio clipping. A good system level compromise will allow some distortion and clipping of sound pressure levels corresponding to the user screaming. This ensures the lower-end of the dynamic range will accept light talking down to whisper level audio.

The placement of the audio port 64 for an air-type microphone 48 is desirably placed on a surface of the mouthpiece 10 which faces the palate, as this position is stable, is exposed to the highest speech sound levels, and does not get impacted by tongue and cheek movements during speech which can give rise to undesired audio artifacts. A tooth-facing position is also an acceptable alternative. It should be noted that due to an in-mouth air-type microphone placement, the user's speech is loud so the signal-to-noise ratio (SNR) is large. External noise sources are dampened not only by the user's own cheeks and dental anatomy, but by the attenuation of the microphone assembly 40. This allows for clear recorded voice audio, down to whisper level, even in very loud external noise environments.

The microphone capability of the mouthpiece may also be implemented using, e.g., a polyvinylidene fluoride or polyvinylidene difluoride (PVDF) film vibrational sensor. This technique utilizes amplification circuitry to process the small signals output from the piezo-electric film material, as well as electrical shielding techniques to help reduce noise. The PVDF film material may be placed on an interior section of the mouthpiece enclosure, to pick up vibrations of the enclosure itself, as long as the film is backed either by air or a flexible material. Benefits of the PVDF film implementation may include a reduced implementation volume as well as inherently good noise shielding from the external environment as air-based incoming sound impinging on the user does not give rise to large physical vibrations inside the user's mouth. Both microphone implementation methods, therefore, provide the ability to issue clear audio transmissions with good SNR even in cases where the user is unable to hear themselves talk due to environmental noise conditions.

The mouthpiece 10 desirably provides a physical vibration drive onto the teeth to implement the bone conduction audio path previously described. This may be accomplished by using a piezo-electric material, e.g., with a piston-type interface to translate the small vibrations onto the surface of the tooth or teeth. Previous disclosures have detailed this interface and potential architecture options, as incorporated herein above, including simple beam with weights to reduce the mechanical resonant frequency, as well as a cantilever type configuration to again help lower the resonant frequency and also reduce the mechanical source impedance of the device to better translate power to the tooth.

Additionally or alternatively, the mouthpiece 10 can have two actuators, located on either side of the mouth (e.g., both located on the left or right side of the mouth, or one (e.g., a first actuator) located on the left side and one (e.g., second actuator) located on the right side), and provide “3D” or “directional” audio by slight timing differences/phasing between the two actuators. This can help to tell the difference between incoming audio from different radio sources in the case where the relay component 12 is connected to multiple infrastructure communication devices 14 at once. Additionally or alternatively, the mouthpiece component 10 can have multiple actuators located on one or both sides of the mouth (e.g., one actuator on a left side and two actuators on a right side or vice versa).

The mouthpiece component 10 can be powered without a battery source. For example, the mouthpiece component 10 can be powered through the oscillations of a magnetic field as a means of live power transfer. For example, the mouthpiece component 10 can be powered with a wireless power transfer system. In this way, the mouthpiece component 10 can be a “passive” component without a battery source. Such a wireless power transfer system may comprise a component dedicated only to transferring power. This component may be located in proximity to the mouthpiece component 10 and other components, when worn by the user such as positioning the power transfer system in a helmet for powering the mouthpiece component 10 and optional earpieces. Alternatively or additionally, the mouthpiece component 10 can have an internal power source. This may either include a replaceable battery or integrated rechargeable battery. The use of a rechargeable battery may allow the system to remain sealed and waterproofed without having to incorporate a separate waterproof compartment to allow for battery replacement. The method of recharging the internal battery may be either contact charging through exposed metal contact pins on the mouthpiece external surface, or through a wireless inductive or resonant charging technique. The wireless methods may allow for complete mouthpiece component sealing and less risk of leaks and fluid ingress. In order to make the inductive charging work for a device small enough to be worn in the mouth, certain design aspects may include creating a very tight coupling between the charger transmit coil and the mouthpiece receive coil. Distance between those coils is desirably minimized through design, and ferrite sheet backing material used to help reduce heat and again increase the coupling coefficient. The device is relatively small enough that a custom transmit coil made to match and align with the mouthpiece recharge coil is advantageous.

The mouthpiece component 10 enclosure itself is desirably physically small but also very strong in order to ensure the internal components and battery are protected and safe even under potential bite-force of the user. To this end, the circuitry and battery may be encapsulated, e.g., with epoxy 54, as part of the design such that all of the potentially hazardous materials are completely encased in a bio-compatible material such as that used in pacemaker headers. A typical implementation would include the epoxy filling 54 of a bio-compatible plastic enclosure, but to reduce size even further the plastic enclosure may be skipped altogether and the epoxy 54 may be molded around the electronics in the desired component shape. This allows for tight control and small dimensions for the gap to a recharge coil, for instance. It also allows for the ability to create a small air-type microphone air cavity 42 through the use of core pins during the epoxy mold process. This air cavity 42 may then be integrated with the aforementioned acoustic attenuation components. Top or bottom port air-type microphones can be used along with this method.

Lastly, FIG. 2C illustrates that the mouthpiece component 10 may be clamped to the user's tooth or teeth, e.g., upper back molars, using some clamping and fit mechanism, as illustrated in the perspective view of the mouthpiece component 60. This is best done by having some portion of the mouthpiece be user-specific, and custom to their dental anatomy, to ensure the best clamp/retaining force, and to optimize comfort. The customized-portion of the mouthpiece may either be integrated into the mouthpiece assembly 10 as a single custom piece, or it may be separable such that the dental-adapter to the individual may be constructed separately than the mouthpiece electronics package, and the two parts mated together to create the overall mouthpiece system component.

FIG. 2C further illustrates that the mouthpiece component 60 may include the microphone assembly housing 62 having a size configured for unobtrusively positioning within the user's mouth for extended periods of time, e.g., along the lingual or buccal surface(s) of one or more teeth, placed against the palate. The housing 62 may have a length of, e.g., 25 mm, and a height of, e.g., 15 mm, with the audio port 64 for an air-type microphone (contained within the housing 62) defined along the side of the housing 62. The audio port 64 may be defined along the side of the housing 62 facing towards the palate when the component 60 is secured within the mouth, but in other variations, the audio port 64 may be defined at other locations such that it faces the teeth (either lingual or buccal surface) or the inner surface of the user's cheek.

In either case, as further illustrated by FIG. 2C, the housing 62 may have a first portion of a conformable securement member 66, e.g., dental acrylic, attached along the side of the housing while a second portion of the conformable securement member 66 may define an interface 68 which is conformable to the patient's underlying dentition. Alternatively, the second portion of the conformable securement member 66 may instead be formed into a simplified configuration which is atraumatic and positionable against a variety of dentition. An actuator housing 70 having a contact portion 72 for placement against the surface(s) of the one or more teeth (e.g., within the interproximal space between teeth) may be positioned in apposition to the interface 68 such that a receiving region 78 is formed between the two within which the one or more teeth may be positioned.

The actuator housing 70 may contain at least some of the electronics and one or more actuators which are configured to vibrate according to the signals received for transmitting auditory signals via vibrational conductance into the underlying surfaces of the one or more teeth in contact with the contact portion 72. The actuator housing 70 and contact portion 72 may be maintained in its position relative to the housing 62 via a connecting member 74, e.g., hypotube, which has an embedded portion 76 secured or otherwise attached within or to conformable securement member 66. The connecting member 74 may be flexible enough to allow the actuator housing 70 and assembly housing 62 to temporarily flex away from one another during securement of the assembly 60 upon the user's dentition as the tooth or teeth are positioned within the receiving region 78.

Yet when suitably positioned, the actuator housing 70 and contact portion 72 may clamp or otherwise become secured against the tooth or teeth, e.g., via an interference fit between the assembly 60 and the surfaces of the user's tooth or teeth. The connecting member 74 may be positioned, e.g., to extend proximally around the distal surface (i.e., opposite of the mesial surface) of the last positioned molar (i.e., third molar) so that the occlusal surfaces of the tooth or teeth remain free and unobstructed by the assembly 60.

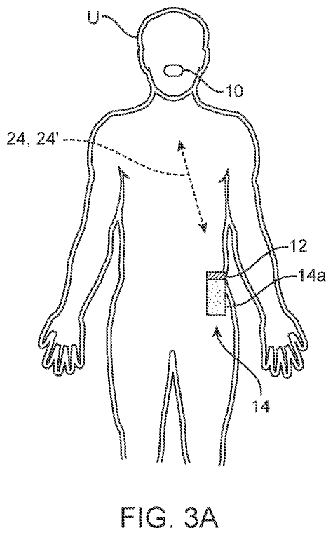

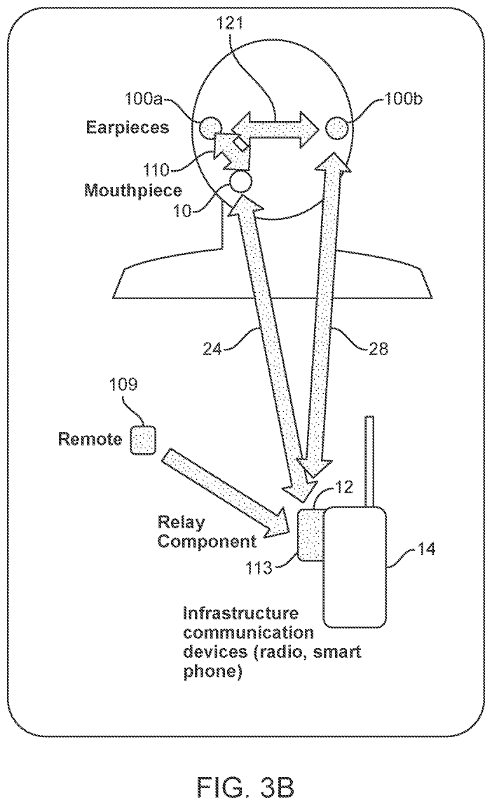

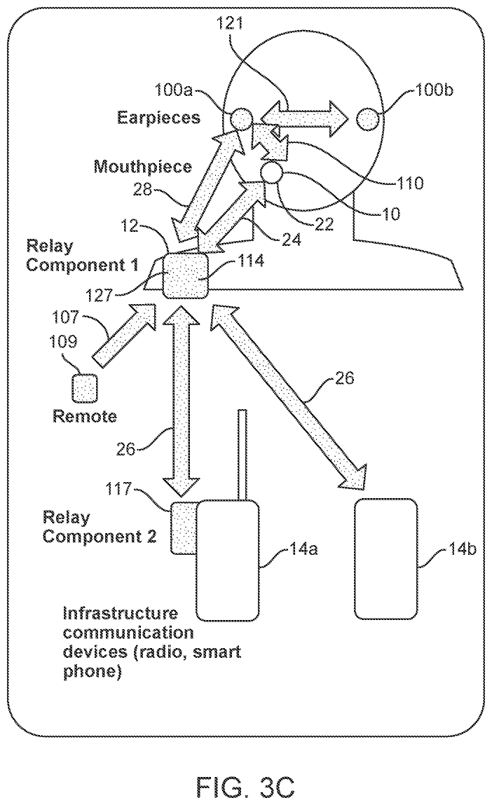

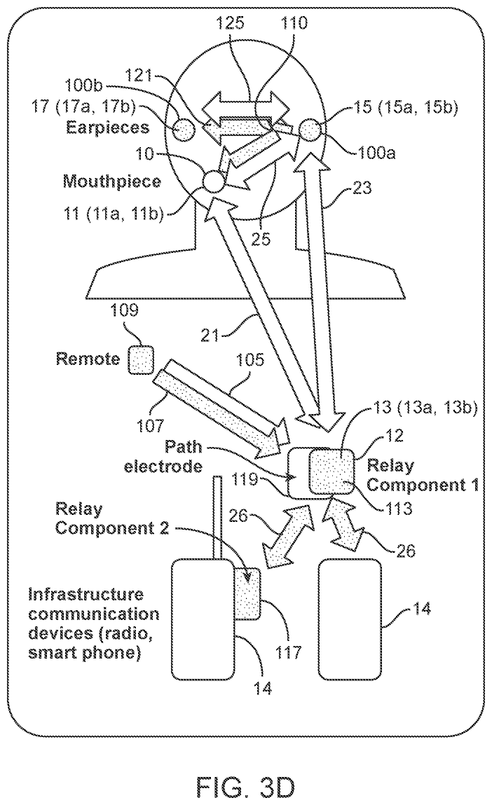

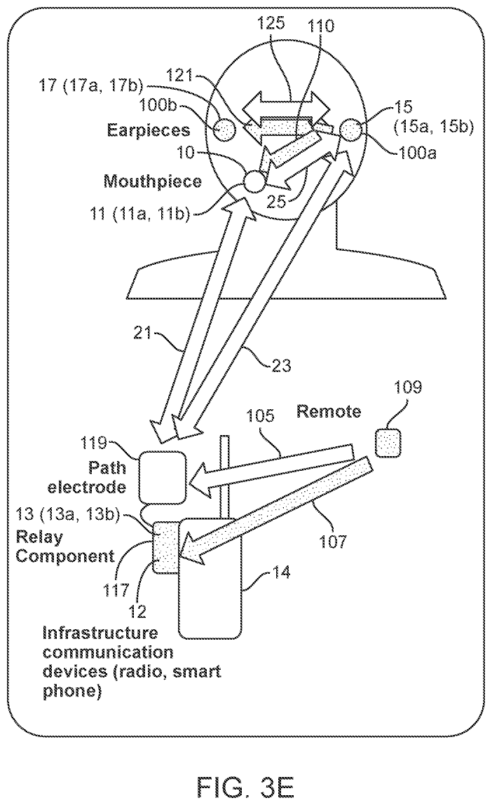

In a variation of the communication system illustrated in FIG. 3A, the relay component 12 may be integrated into the infrastructure component 14a, e.g., directly integrated, integrated into a case or housing such as a cell phone case, etc., such that a single integrated infrastructure communications device 14 may be used to communicate via the wireless link 24 with the mouthpiece component 10. Therefore, the relay component 12 of the communication system can function as an interface between the infrastructure communications device 14 (e.g., cell phone, walkie-talkie radios, intercom device, etc.) and the mouthpiece component 10. The relay component 12 may convert the audio available at the infrastructure communication device into a wireless signal expected by the mouthpiece component 10, and vice-versa.

Depending on the wireless link type chosen between the mouthpiece component 10 and relay component 12, possible wireless links 24 between the mouthpiece component 10 and relay component 12 may include, e.g., Near Field Magnetic Induction (NFMI), or WBAN technology including human body-conduction or RF such as NB, UWB or Bluetooth® or a combination of these. One link type is NFMI which enjoys relatively little attenuation through body tissue, and also has a low electromagnetic propagation for a low RF profile. For this implementation, the relay component 12 can incorporate an inductive coil component which may be incorporated into the communications system.

The wireless link 24 between the relay component 12 and the mouthpiece component 10 may be encrypted to ensure secure communications, e.g., using AES-256 or another chosen encryption algorithm. If unique encryption key-exchange is required, this may be achieved through an exchange over NFMI, NFC, or other close proximity-based method, between the mouthpiece component 10 and desired relay component 12, prior to use.

Additionally, audio data transmitted between the infrastructure communication device 14, the relay component 12, the mouthpiece component 10, and/or the optional earpiece components 100 can be compressed to fit into a data link bandwidth, such as using a 16 kHz sample rate at 12-bit resolution and 64 kbps which provides 3:1 compression and good quality audio while being compatible with any of the WBAN or magnetic options. The audio data can further be encrypted or decrypted at the transmitter and/or receiver or the transceiver 22 to provide a secure wireless link. This encryption can use security functions described in, e.g., FIPS 140-2 Annex A, including AES or TDEA or other methods. The mouthpiece component 10, the relay component 12, an adapter/secondary relay component 117, and/or the optional earpiece components 100 can all be responsible for encrypting and decrypting. Bluetooth communication between the relay component 12 and a smartphone 14 can also utilize built-in encryption.

Audio bandwidth and signal encryption and management of the mouthpiece component 10 and earpiece components 100 (for example, communication between mouthpiece component 10 and earpiece components 100 or between earpiece components 100 using time-sharing or frequency channel selection) for the system illustrated in FIG. 3B can be consistent with the system illustrated and described in any of the following figures.

While the physical implementation of the relay component 12 may be a separate electronics enclosure with attached wireless link antenna to communicate with the mouthpiece component 10 and a 2nd relay component 117, other variations may have the relay component 12 physically mate and/or integrate with the infrastructure communication device 14 (described in further detail below). Since both devices would be outside of the body of the user, there are less restrictions on this link implementation, and protocols such as Bluetooth® or other RF communications may be used.

Regardless of the specific chosen placement and implementation of the relay component 12, from a system level, the relay component 12 may incorporate one or more processors for implementing digital signal processing (DSP) in order to minimize power consumption at the mouthpiece component 10. For a half-duplex system the DSP is not as critical as there is no feedback issue, but even basic filtering may be done at this point to save power.

The relay component 12 may either obtain power directly from the infrastructure communication device (if available), or can be battery powered and require either battery replacements or recharge. An integrated rechargeable battery may be implemented for size reduction reasons. In order to save power, when implementing a half-duplex system over a full-duplex infrastructure such as a cell phone, an audio-level sensor may be used to notify the system when it is able to transition into an idle mode (not actively transmitting or receiving audio). Moreover, the relay component 12 may incorporate one or more user interface features which allow for the user U to interface with the system settings and modes, e.g., a push-to-talk (PTT) button for half-duplex audio communications and/or full-duplex communications (or to switch between half- and full-duplex communications), receive volume controls, audio routing options (e.g., to mouth and/or ears, selection of side etc.), etc.

FIG. 3B further illustrates how the mouthpiece component 10 and the optional earpiece components 100a, 100b can communicate directly with the relay component 12, which can adapt to an analog input of the infrastructure device 14. The link between the mouthpiece component 10, the earpiece components 100a, 100b, and the relay component 12 may utilize NFMI or NB (MICS or ISM 433M Hz bands) as described previously due to its relative tolerance of tissue in the transmission path. Alternatively, the wireless link from the relay component 12 to one or more of the earpiece components may include a base-band NFMI (not modulated at a higher frequency), as described in further detail below, which is a non-encrypted version but can be made to work with a number of conventional earpiece components. Use of base-band NFMI may allow for a relatively small range for sniffing (non-propagating).

FIG. 3B further illustrates that the wireless remote control 109 could wirelessly link to the relay component 12 via a wireless link 107, utilizing NFMI or any of the WBAN technologies since the connection is outside the body, and can also relay through a second radio transceiver 113 in the relay component 12. An NFMI link which connects the wireless remote control 109 with the relay component 12 can desirably allow for better performance, e.g., under water. For the wireless remote control 109, the implementation may be something physically similar to, e.g., a key-fob, to be worn in the pocket for easy concealment, or more advanced devices could also provide system status information, such as sending data to/from a smart watch.

System controls 16, e.g. for a PTT functionality or volume control, may be handled by several methods including physical controls on the relay component 12, a wireless remote control 109, voice activated commands, and/or button actuation on the mouthpiece component 10 and/or optional earpiece components 100. System controls 16 may be integrated into the physical implementation of the relay component 12, or they may be separated out into a separate wireless remote control 109 with a new wireless data link, such as Bluetooth® (e.g., Bluetooth® low-energy (BLE)), other RF link, an NFMI link, or simply a wired connection. Voice commands can include voice activation (VAD) based on signal energy content or spectral content to enable outgoing communication from the mouthpiece component 10 and/or for the system controls 16 for, e.g., volume adjustment based on spectral content of verbal commands or the system controls 16 based on codes, e.g., tooth clicks. Incorporation of one or more sensors (e.g., membrane or dome switches or capacitive sensing) on the mouthpiece component 10 can allow tongue actuation for the system controls 16.

FIG. 3B further illustrates wireless data links 110, 121 between the mouthpiece component 10 and the first earpiece component 100a and/or between the earpiece components 100a and the second earpiece component 100b, respectively. Wireless data links 110, 121 can utilize either NFMI or the NB (MICS or 433-434 MHz) ISM band due to the relative tolerance of tissue in the transmission path. The relay component 12 can utilize the wireless communication links 24, 28, such as an NFMI link or using NB (MICS or ISM 433M Hz bands) as described previously.

The mouthpiece component 10 and/or the optional earpiece components 100a, 100b can communicate with the relay component 12 or each other to achieve intelligent or selectable audio routing to ears or mouth or both as well as adaptive noise reduction. The relay component 12 can link with the mouthpiece component 10 alone, the mouthpiece component 10 and/or the optional earpiece components 100a, 100b using the NFMI link, assuming sufficient parallel audio streams, e.g.. NXP Nx2280, having a capability of up to three parallel streams in which one audio stream can be routed to the mouthpiece component 10 and a second stream can be routed to both ears, or one to the left ear and one to the right ear via the optional earpiece components 100a, 100b.

FIG. 3C illustrates that the relay component 12 can be shoulder-mounted with an optional push-to-talk (PTT) control 127 that uses the wireless communication links 24, 28, such as an NFMI link or using NB (MICS or ISM 433M Hz bands) as described previously, to communicate with the mouthpiece 10 and/or earpiece components 100a, 100b. If using NFMI, the mouthpiece component 10 and optional earpiece components 100 can be separate devices on a network that supports multiple nodes and parallel audio streams (e.g., NXP NxH2280) or on separate NFMI frequency channels.

The relay component 12 can be configured to be placed near the user's head to allow a reliable inductive link to the mouthpiece component 10 and/or the optional earpiece components 100a, 100b on the head and at a natural position for the user to grasp and handle (e.g. PTT or volume controls). The link distance and variable positioning of the head relative to the relay component 12 can be addressed with multiple selectable antennas in the relay component 12. Alternatively, a single antenna may be used when incorporated, e.g., as a loop around the neck of the user. Due to the nature of the field line directions, a single antenna may allow coverage for full head movement of the user. In another alternative, a single location antenna may be used, e.g., located at the back of the user's head or on a helmet. A single coil/antenna can be in the mouthpiece component 10 and/or the optional earpiece components 100 and one or multiple coil or loop antennas can be included in the relay component 12. Improved link margin, allowing for the extended link distance and variable positioning of the head relative to the relay component 12, can be addressed with multiple selectable coils/antennas in the relay component 12 can be achieved by using orthogonal coils or loops (e.g., 3-axis) and dynamically adapting the transceiver 113 of the relay component 12 to one of the multiple selectable coils/antennas with the highest signal strength, which allows for head movement and changes in position of the mouthpiece component 10.

Wireless data links 110, 121 between the mouthpiece component 10 and the earpiece components 100a and/or between the two earpiece components 100a, 100b can utilize either NFMI or using NB (MICS or ISM 433M Hz bands) as described previously due to its relative tolerance of tissue in the transmission path. Additionally, the wireless link 107 between the wireless remote 109 and the relay component 12 can utilize NFMI or any WBAN wireless link since the connection is outside the body.

FIG. 3C further illustrates that the relay component 12 can further comprise a second radio transceiver 114 for downstream communication with the infrastructure communication device 14. This second radio transceiver 114 can utilize any of the NB or WBAN options described above with an appropriate antenna or it can utilize a second NFMI frequency with coil(s) or loop antenna(s). Alternatively, the relay component 12 can utilize a single NFMI radio transceiver and a set of coils/loop antennas and simultaneously link to both the infrastructure communication device 14 and the mouthpiece component 10 using a radio capable of multiple nodes and simultaneous transmit/receive paths such that the single NFMI radio transceiver, e.g., second radio transceiver 114 may link to all components. The relay component 12 can be self-powered, containing replaceable or rechargeable batteries, e.g., rechargeable lithium ion.

FIG. 3C further illustrates that wireless communication with the infrastructure communication device 14 can utilize native communication links (e.g. Bluetooth for a smartphone 14b) or an adapter/secondary relay component 117 that is physically attached to the infrastructure component 14a, e.g., a radio or smartphone, that interfaces an analog audio, e.g., a microphone or speaker, signal to the required radio transceiver. This adapter 117 can include antenna/antennas to link with the relay component 12. The wireless data link 26 between the relay component 12 and the adapter/secondary relay component 117 can utilize either NFMI or any WBAN wireless link since the connection is outside the body. For example, for a NFMI transmission, one or more set of orthogonal coils/loop antennas can be utilized in the relay component 12 to allow dynamic selection of an antenna with highest signal strength for a fixed coil/loop antenna in the adapter/secondary relay component 117. Furthermore, a second WBAN transceiver which may be incorporated within relay component 12 may communicate over wireless data link 26 to the adapter/secondary relay component 117 or to infrastructure communication device 14.

FIG. 3D illustrates that the mouthpiece component 10, the optional earpiece components 100, and/or the relay component 12 can communicate wirelessly over body conduction via a body conduction data link 21 (between the relay component 12 and the mouthpiece 10), a body conduction data link 23 (between the relay component 12 and one of the earpiece components 100), and/or a body conduction data link 25 (between the mouthpiece component 10 and one of the earpiece component 100), utilizing either eHBC or mHBC, which can be coupled through a patch electrode/antenna 119, either directly (e.g. embedded) or via a short wire, through the relay component 12. The patch electrode/antenna 119 can be either a galvanic or capacitive electrode to support eHBC or contain a coil or loop antenna for mHBC. For mHBC, the relay component 12 can contain one or more coil/loop antenna or transceiver(s) that can be positioned close to the body (e.g. in a pocket or under clothing) without using the patch 119, as further described below. The relay component 12 can then process the audio data to/from the mouthpiece component 10 and optional earpiece components 100 (e.g. encrypts/decrypts, (de)compresses, applies filtering or gain) and can interface to the adaptor or second radio transceiver 113 which wirelessly links to downstream infrastructure devices 14, which may be over NFMI or any of the WBAN technologies discussed previously. The second relay component 117 can adapt to the analog audio interface of the radio or phone if a wireless protocol is not handled natively.

FIG. 3D further illustrates that for the body conduction wireless link 21, the mouthpiece component 10 can further comprise a first coil 11 (also referred to as first antenna, first coil antenna, first transceiver, or variants thereof) configured to produce a first magnetic field through the user's body. The first coil 11 can comprise a first transmitting coil 11a, configured to produce the first magnetic field through the user's body, and a first receiving coil 11b configured to resonantly couple with other magnetic fields. While the first transmitting coil 11a and first receiving coil 11b may be separate coils, in other variations coils 11a and 11b may comprise the same coil which serves both transmitting and receiving functions. The relay component can further comprise a second coil 13 (also referred to as second antenna, second coil antenna, second transceiver, or variants thereof) configured to produce a second magnetic field through the user's body. The second coil 13 can comprise a second transmitting coil 13a, configured to produce the second magnetic field through the user's body, and a second receiving coil 13b configured to resonantly couple with other magnetic fields.

The body conduction data link 21 can be formed between the first coil 11 and the second coil 13, wherein the first and second coils 11, 13 are configured to resonantly couple with one another when the magnetic fields are propagated through tissue of the user's body for wireless communication for sending audio or data from the mouthpiece component 10 to the relay component 12 and/or vice versa.

If the first coil 11 comprises the first transmitting coil 11a and the first receiving coil 11b and the second coil 13 comprises the second transmitting coil 13a and the second receiving coil 13b, the body conduction data link 21 can be formed between the first transmitting coil 11a and the second receiving coil 13b, wherein the second receiving coil 13b is configured to resonantly couple with the first transmitting coil 11a when the magnetic fields are propagating through tissue of the user's body for wireless communication for sending audio or data from the mouthpiece component 10 to the relay component 12. The body conduction data link 21 can also be formed between the second transmitting coil 13a and the first receiving coil 11b, wherein the first receiving coil 11b is configured to resonantly couple with the second transmitting coil 13a when the magnetic fields are propagating through tissue of the user's body for wireless communication for sending audio or data from the relay component 12 to the mouthpiece component 10.

FIG. 3D further illustrates that the two earpiece components 100a, 100b can communicate with one another via the wireless link 121 or via a body conduction wireless link 125, such as either eHBC or mHBC. Additionally, the wireless link 107 between the wireless remote 109 and the relay component 12 can utilize NFMI or any WBAN wireless link since the connection is outside the body or a body conduction wireless link 105 between the wireless remote 109 and the relay component 12, such as utilizing either eHBC or mHBC.

FIG. 3D further illustrates that for the body conduction wireless link 23, the first earpiece components 100a can further comprise a third coil 15 (also referred to as third antenna, third coil antenna, third transceiver, or variants thereof) configured to produce a third magnetic field through the user's body. The third coil 15 can comprise a third transmitting coil 15a configured to produce the third magnetic field through the user's body and a third receiving coil 15b configured to resonantly couple with other magnetic fields.

The body conduction data link 23 can be formed between the second and third coils 13, 15, wherein the second and third coils 13, 15 are configured to resonantly couple with one another when the magnetic fields are propagated through tissue of the user's body for wireless communication for sending audio or data from the mouthpiece component 10 to the relay component 12 and/or vice versa.

If the second coil 13 comprises the second transmitting coil 13a and the second receiving coil 13b and the third coil 15 comprises the third transmitting coil 15a and the third receiving coil 15b, the body conduction data link 23 can be formed between the second transmitting coil 13a and the third receiving coil 15b, wherein the third receiving coil 15b can resonantly couple with the second transmitting coil 13a when the magnetic fields are propagating through tissue of the user's body for wireless communication for sending audio or data from the relay component 12 to the first earpiece component 100a. The body conduction data link 23 can also be formed between the third transmitting coil 15a and the second receiving coil 13b, wherein the second receiving coil 13b is configured to resonantly couple with the third transmitting coil 15a when the magnetic fields are propagating through tissue of the user's body for wireless communication for sending audio or data from the first earpiece component 100a to the relay component 12.

FIG. 3D further illustrates that the body conduction data link 25 can be formed between the first and third coils 11, 15, wherein the first and third coils 11, 15, are configured to resonantly couple with one another when the magnetic fields are propagated through tissue of the user's body for wireless communication for sending audio or data from the mouthpiece component 10 to the first earpiece component 100a.

If the first coil 11 comprises the first transmitting coil 11a and the first receiving coil 11b and the third coil 15 comprises the third transmitting coil 15a and the third receiving coil 15b, the body conduction data link 25 can also be formed between the third transmitting coil 15a and the first receiving coil 11b, wherein the first receiving coil 11b is configured to resonantly couple with the third transmitting coil 15a when the magnetic fields are propagating through tissue of the user's body for wireless communication for sending audio or data from the first earpiece component 100a to the mouthpiece component 10.

FIG. 3D further illustrates that for the body conduction wireless link 125, the second earpiece components 100b can further comprise a fourth coil 17 (also referred to as fourth antenna, fourth coil antenna, fourth transceiver, or variants thereof) configured to produce a fourth magnetic field through the user's body. The fourth coil 17 can comprise a fourth transmitting coil 17a configured to produce the fourth magnetic field through the user's body and a fourth receiving coil 17b.

If the fourth coil 17 comprises the fourth transmitting coil 17a and the fourth receiving coil 17b, the body conduction data link 125 can be formed between the third transmitting coil 15a and the fourth receiving coil 17b, wherein the fourth receiving coil 17b can resonantly couple with the third transmitting coil 15a when the magnetic fields are propagating through tissue of the user's body for wireless communication for sending audio or data from the first earpiece component 100a to the second earpiece component 100b. The body conduction data link 125 can also be formed between the fourth transmitting coil 17a and the third receiving coil 15b, wherein the third receiving coil 15b is configured to resonantly couple with the fourth transmitting coil 17a when the magnetic fields are propagating through tissue of the user's body for wireless communication for sending audio or data from the second earpiece component 100b to the first earpiece component 100a.

In each of these embodiments, the inductive coils 11, 13, 15 and 17 may operate at the same frequency, thereby time-sharing or coordinating the data link. The body conduction wireless links 21, 23, 25, 105, 125 can each operate at a different frequency from one another, or the body conduction wireless links for each can be time-shared/coordinated with each other such that a different frequency is not needed.

The communication system illustrated in FIG. 3E is similar to the communication system illustrated in FIG. 3D. FIG. 3E further illustrates that the mouthpiece component 10, optional earpieces components 100a, 100b, and/or the relay component 12 can communicate wirelessly over the body conduction wireless links 21, 23, 25, 105, 125 (either eHBC or mHBC) that can directly adapt the signal between the patch electrode/antenna 119 (galvanic or capacitive for eHBC or containing a loop/coil antenna for mHBC) and the analog audio interface of the infrastructure communication device 14 without intervening relay component 12. For eHBC, the patch electrode/antenna 119 can be positioned on the skin and connected by a short wire to the relay component 12. Additionally in the case of eHBC, the body conduction wireless links 21, 23, 25, 105, 125 may be accomplished alternatively using galvanic or capacitive electrodes rather than inductive coils. For mHBC, the relay component 12 can comprise the patch antenna 119 having the second coil 13, and can be positioned near the body, such as attached to body worn equipment or in a pocket, or the relay component 12 can comprise the second coil 13 without the need for a separate patch 119.

System controls 16 can be handled by the wireless remote 109 using the wireless link 107 utilizing NFMI or any of the WBAN technologies. Alternatively, the remote control 109 may use the body conduction wireless link 105 utilizing either eHBC or mHBC to establish a low power, low detectability wireless link to the relay component 12.

As describe above, the system can optionally include an earpiece component 100. For example, FIG. 4 illustrates that the two-way communication system can have one or multiple earpiece components 100 (e.g., 1, 2, 3, 4, 5, 6, 7, 8, 9, or 10 or more earpieces 100). When more than two earpieces 100 are used, these can be designed and used as back-up or replacement earpieces 100 to the one or two primary earpieces 100. The earpieces 100 are also referred to as earpiece components 100 throughout. The system can have a left earpiece component 100a and/or a right earpiece component 100b. As another example, the system can have one or multiple left earpiece components 100a (e.g., 1, 2, 3, 4, or 5 or more left earpiece components 100a) and/or can have one or multiple right earpiece components 100b (e.g., 1, 2, 3, 4, or 5 or more right earpiece components 100b), where the number of left and/or right earpiece components 100 beyond the first earpiece 100a or 100b on each side, i.e., the second, third, fourth, and fifth left and/or right earpieces 100, can be back-up or replacement earpieces 100 for the first and/or second earpiece components 100a, 100b. Each earpiece component 100 can be configured to be a communication device. Each earpiece component 100 can be capable of one-way or two-way communication. The system can provide half-duplex and/or full duplex communications. Each earpiece component 100 can be an assembly of multiple sub-elements that when assembled comprise an earpiece component 100.

Having multiple earpiece components 100 (e.g., 2 or more) can advantageously provide communication redundancy. Should an earpiece 100 malfunction or be destroyed during use, a user can quickly don a fully functional earpiece 100 with minimal delay and/or rely on the other functional earpiece component 100 in the other ear. Each earpiece component 100 can have one or multiple communication channels configured to transmit and/or receive sound at one or multiple frequencies, for example, behaving as base-band audio for analog variations or modulating higher frequencies and work digitally as well. The left and right earpieces 100a, 100b can have the same or different frequency channels relative to one another.

Each earpiece component 100 can be removably engageable with/to a user's ear. For example, the earpiece components 100 can be configured for temporary securement on/to a user's ears. One or more of the earpiece components 100 can be configured to fit partially or completely in an ear, for example, in at least a portion of an ear canal. For example, the earpiece components 100 can extend into about 1% to about 100% a length of the ear canal, for example, about 25%, about 33%, about 50%, about 67%, about 75%, or about 100% the full or half length of the ear canal. One or more of the earpiece components 100 can be configured to fit partially or completely over or behind an ear, for example, over at least a portion of an outer ear. For example, the earpiece components 100 can extend over about 1% to about 100% a surface of the outer ear, for example, about 25%, about 33%, about 50%, about 67%, about 75%, or about 100% the surface of the outer ear. In this way, each earpiece component 100 can be an in-the-ear earpiece component and/or an over-the-ear and/or a behind-the-ear earpiece component. The earpiece components 100 can have the same or different configurations from one another. For example, the left and right earpiece components 100a, 100b can both be an in-the-ear earpiece component and/or an over-the-ear earpiece component and/or a behind-the-ear earpiece component. As another example, the left earpiece component 100a can be an in-the-ear earpiece component and the right earpiece component 100b can be an over-the-ear or behind-the-ear earpiece component, or vice versa.

Each earpiece component 100 can be configured to fit in and/or on a left ear, a right ear, and/or both ears (separately or together). The earpiece components 100 can be foamies, can have tiered shapes, can have tapered shapes, can be custom in-ear inserts, can be ear-cups, configured to be ear-side specific (left or right), shaped such that they can be placed in either ear, or any combination thereof. The earpiece components 100 can be capable of, adapted to, and/or configured to receive sound. For example, foamies (and other material only earpiece components) can be capable of, adapted to, and/or configured to receive sound. The earpiece components 100 can be made from foam, silicone, acrylic, vinyl, rubber, plastic, or any combination thereof. One or more portions of the earpiece components 100 can be compressible and/or one or more portions of the earpiece components 100 can be incompressible. For example, electronic components in the earpieces 100 can be incompressible and the material(s) which encase the electronic components can be compressible and/or incompressible. The earpiece components 100 can have one or multiple noise reduction flanges, for example, 1-10 noise reduction flanges (e.g., 1, 2, 3, 4, 5, 6, 7, 8, 9, or 10 or more noise reduction flanges). The earpiece components 100 can be vented or non-vented. The one or more vents in a vented earpiece component 100 can have a vent opening and/or a passageway that has an adjustable or fixed geometry (e.g., the size of the vent opening and/or passageway can be increased and decreased). The earpiece components 100 can be disposable or reusable. The earpiece components 100 can be battery powered and/or wirelessly powered. The earpiece components 100 can have a rechargeable power source. The rechargeable power source can be recharged wirelessly or via direct contact with a recharge source. A connector can connect two or more earpieces 100 together. The connector can be an electrical connector (e.g., an insulated wire) or a non-electrical connector (e.g., a chord, a string, etc.).

The earpiece components 100 can be optionally configured to communicate with the environment, a user (e.g., one or both ears), one or more components of the system, or any combination thereof. The earpiece components 100 can be configured to receive and/or transmit sound and/or electronic signals that represent sound (also referred to as electronic sound signals, sound signals, audio signals, or variants thereof). For example, the earpiece components 100 can be configured to receive/transmit communications (e.g., sound waves and/or audio signals) from/to the environment, from/to the mouthpiece component 10, from/to the relay component 12, or any combination thereof. For example, the earpiece components 100 can receive audio signals from the environment and the relay component 12, and then transmit these received signals to the ear canal. In this way, the earpiece component 100 can be a one-way communication component such that the earpiece component 100 can provide incoming audio but not outgoing communications. For example, FIG. 4 illustrates that the earpiece components 100 can be configured to receive external sound 102 from the environment (also referred to as environmental sound or environmental noise), receive audio signals from the mouthpiece component 10, receive audio signals from the relay component 12, or any combination thereof. The sound and/or electronic sound signals that the earpiece components 100 receive is collectively referred to as incoming sound.

FIG. 4 further illustrate that each earpiece component 100 can have one or more earpiece microphones 104 and/or one or more earpiece speakers 106. The one or more microphones 104 can each be configured to pick up environmental sound 102 and they may also be configured to face into the ear canal and pick up in-head vibrations from the user speaking. The picked up environmental sound 102 can be communicated to a user with or without being processed and/or with or without first (or at all) being converted into an electronic signal. Each of the one or more speakers 106 can be configured to emit sound received from an earpiece microphone 104, to emit sound that corresponds to the audio signals received from the mouthpiece component 10, to emit sound that corresponds to the audio signals received from the relay component 12, to cancel environmental sound 102, or any combination thereof.

FIG. 4 further illustrates that each earpiece component 100 can have a transmitter and/or a receiver such as an earpiece transceiver 108. The earpiece transceiver 108 can enable the earpiece components 100 to be in wireless communication with the mouthpiece component 10 and/or the relay component 12. The earpiece transceiver 108 can enable the earpiece components 100 to receive and/or transmit audio data from and/or to the mouthpiece component 10 and/or the relay component 12. FIG. 4 further illustrates that the system can have the wireless data link 28 between the earpiece components 100 and the relay component 12 (the wireless data link 28 is also referred to as a relay component data link 28, communication link 28, or variants thereof). For example, the data link 28 can be established between the earpiece components 100 and the antenna/coil of the relay component 12. The data link 28 can electrically connect the earpiece components 100 to the relay component 12. The data link 28 can be used to get audio data to and/or from relay component 12. Audio signals can be transmitted over the data link 28. Control signals 16 can be transmitted over the data link 28. For example, the system can generate control signals 16 that can lower environmental audio levels when incoming audio is present. The control signals 16 can be associated with one or more controls. The controls, when toggled or otherwise controlled, can generate one or more control signals 16. The system can have one or more data links 28, for example, one, two, or more data links 28. For example, the system can have a first data link 28a between a first earpiece component 100a and the relay component 12 and a second data link 28b between a second earpiece component 100b and the relay component 12. The first and second earpiece components 100a, 100b can be left and right earpiece components, respectively, and the first and second data links 28a, 28b can be left and right data links, respectively. As another example, the system can have a single data link 28 between the left and right earpiece components 100a, 100b and the relay component 12.

FIG. 4 further illustrates that the system can have the data link 107 between the relay component 12 and a wireless remote 109 (the data link 107 is also referred to as a wireless remote data link 107, communication link 107, or variants thereof). For example, the data link 107 can be established between the wireless remote 109 and the antenna/coil of the relay component 12. The data link 107 can electrically connect the wireless remote 109 to the relay component 12. The data link 107 can be used to send data to the relay component 12 from the wireless remote 109. System control 16 can send signals to be transmitted over the data link 107 via the wireless remote 109. For example, the system can generate control signals 16 that toggle between how the other component communicate, e.g., toggle between sending communication from the mouthpiece component to the relay component to sending communication from the relay component to the mouthpiece component. The control signals 16 can be associated with one or more controls. The controls, when toggled or otherwise controlled, can generate one or more control signals 16. The system can have one or more data links 107, for example, one, two, or more data links 107a, 107b. For example, the system can have a first data link 107a between a first wireless remote 109a and the relay component 12 and a second data link 107b between a second wireless remote 109b and the relay component 12, wherein the first and second data links 107a, 107b are associated with different controls.

FIG. 4 illustrates that the system can have a wireless data link 110 between the earpiece components 100 and the mouthpiece component 10 (the data link 110 is also referred to as a mouthpiece data link 110, communication link 110, or variants thereof). For example, the data link 110 can be established between the earpiece components 100 and the one or more coils 11 of the mouthpiece component 10. One benefit of using the mouthpiece component 10 to earpiece components 100 wireless data link 110 is that they are quite close to each other and in a relatively fixed orientation allowing the use of a directional magnetic field antenna. Then the earpiece components 100 can relay the mouthpiece data down to the relay component 12 or to the infrastructure communication device 14 in which case the earpiece components 100 may act as a relay component. The data link 110 can electrically connect the earpiece components 100 to the mouthpiece component 10. The data link 110 can be used to get audio data to and/or from mouthpiece component 10. Audio signals can be transmitted over the data link 110. Control signals 16 can be transmitted over the data link 110 and/or over a separate data link 111. For example, the control signals 16 can directly interface with the earpiece components 100 via the data link 110 and/or via the separate data link 111 in communication with the data link 110 as shown in FIG. 4. The system can have one or more data links 110, for example, one, two, or more data links 110. For example, the system can have a first data link 110a between a first earpiece component 100a and the mouthpiece component 10 and a second data link 110b between a second earpiece component 100b and the mouthpiece component 10. The first and second earpiece components 100a, 100b can be left and right earpiece components, respectively, and the first and second data links 110a, 110b can be left and right data links, respectively. As another example, the system can have a single data link 110 between the left and right earpiece components 100a, 100b and the mouthpiece component 10.

The system can have one or more relay component data links 28 (e.g., first and second relay component data links 28a, 28b; left and right relay component data links 28a, 28b) and/or one or more mouthpiece component data links 110 (e.g., first and second mouthpiece component data links 110a, 110b; left and right mouthpiece component data links 110a, 110b).

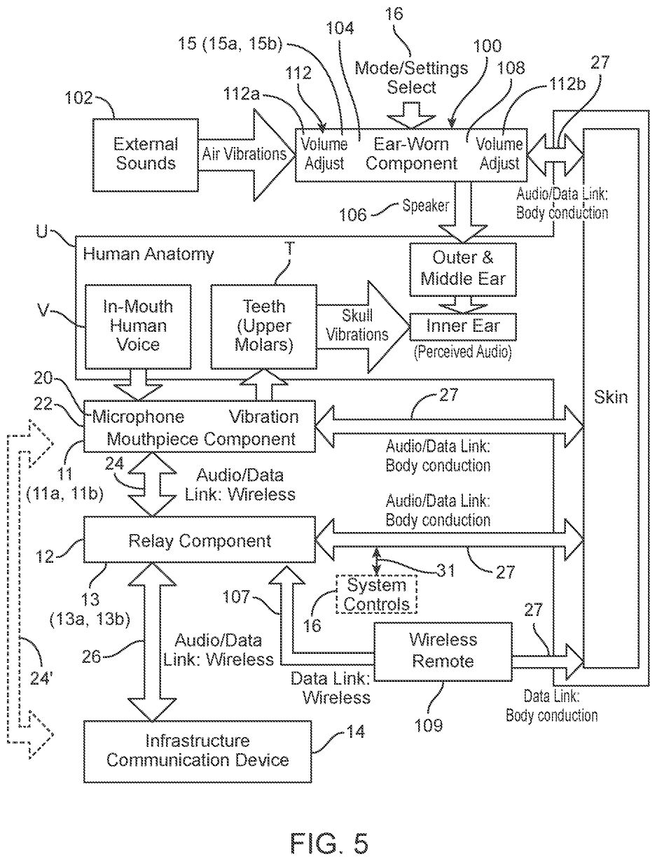

FIG. 4 illustrates that the system controls 16 can interface with the relay component 12 via the data link 28. For example, the system controls 16 can interface directly with the data link 28 and/or via a separate systems control data link 29 in communication with the data link 28 as shown in FIG. 4 and/or interface directly with audio/data link via body conduction 25 and/or via a separate systems control data link 31 as shown in FIG. 5. Additionally or alternatively, the system controls 16 can interface with the mouthpiece component 10 via the data link 110. The system controls 16 can be electrically linked to the earpiece components 100, the mouthpiece component 10, the relay component 12, or any combination thereof. For example, FIG. 4 illustrates that the system controls 16 can be in communication with the relay component 12 and the earpiece components 100 via the separate systems control data link 29.

The communication link 28 between the earpiece components 100 and the relay component 12 can utilize any number of wireless data transmission protocols, for example, NFMI (digital and/or analog, with analog being, for example, either modulated or base-band audio (e.g., tele-coil)), human body conduction, other WBAN RF wireless platforms (e.g., BT, BLE, custom ISM band), or any combination thereof. For example, the earpiece components 100 can be configured to wirelessly receive/transmit audio communications from/to the relay component 12 and/or the antenna/coil of the relay component 12 using analog NFMI. An analog NFMI protocol can utilize a base audio band and an inductively coupled signal. As another example, the earpiece components 100 can be configured to wirelessly receive/transmit audio communications from/to the mouthpiece component 10 or relay component 12 using digital NFMI. Relative to analog NFMI, digital NFMI can allow for better audio control and encryption possibilities. For digital implementations of the communication link 28, the data (e.g., audio data) communicated between the earpiece components 100 and the relay component 12 can be encrypted to ensure security using, for example, AES-256 or other encryption standards.

The communication link 110 between the earpiece components 100 and the mouthpiece component 10 can utilize any number of wireless data transmission protocols, for example, NFMI (digital and/or analog), NB (MICS, ISM 433 MHz), body conduction, other wireless platforms, or any combination thereof. For example, the earpiece components 100 can be configured to wirelessly receive/transmit audio communications from/to the mouthpiece component 10 using analog NFMI (e.g., with sensitive and noise free circuitry). An analog NFMI protocol can utilize a base audio band and an inductively coupled signal. As another example, the earpiece components 100 can be configured to wirelessly receive/transmit audio communications from/to the mouthpiece component 10 using digital NFMI. Relative to analog NFMI, digital NFMI can allow for better audio control and encryption possibilities. For digital implementations of the communication link 110, the data (e.g., audio data) communicated between the earpiece components 100 and the mouthpiece component 10 can be encrypted to ensure security using, for example, AES-256 or other encryption standards.

One or multiple earpiece components 100 can be configured with analog and digital NFMI for wireless communication with the relay component 12, the antenna/coil of the relay component 12, the mouthpiece component 10, the mouthpiece coil(s) 11, or any combination thereof. This can advantageously provide communication redundancy in the event of equipment malfunction. Digital NFMI can be the default wireless communication protocol and analog NFMI can be the backup wireless communication protocol, or vice versa. The analog NFMI and digital NFMI communication links can be simultaneously or separately activated and/or deactivated. For example, the analog NFMI link can be configured to be activated only when the digital NFMI link is turned off, is not functional, or has an error rate greater than a threshold error rate, or vice versa. Additionally or alternatively, when the analog and digital NFMI communication links are simultaneously activated, the earpiece components 100 can be configured to wirelessly communicate with the relay component 12, the antenna/coil of the relay component 12, the mouthpiece component 10, the mouthpiece coil(s) 11, or any combination thereof using digital NFMI but not analog NFMI unless the digital NFMI link is turned off, is not functional, or has an error rate greater than a threshold error rate, or vice versa. A user can manually toggle between the analog and digital NFMI protocols and/or the system can automatically toggle between the analog and digital NFMI protocols, for example, upon detecting that communications have degraded.

The left earpiece component 100a can be configured to wirelessly communicate with the relay component 12, the antenna/coil of the relay component 12, the mouthpiece component 10, the mouthpiece coil(s) 11, or any combination thereof using analog NFMI and the right earpiece component 100b can be configured to wirelessly communicate with the relay component 12, the antenna/coil of the relay component 12, the mouthpiece component 10, the mouthpiece coil(s) 11, or any combination thereof using digital NFMI, or vice versa.

The left earpiece component 100a can be configured to be in wired communication with the relay component 12 and/or the antenna/coil of the relay component 12 and the right earpiece component 100b can be configured to wirelessly communicate with the relay component 12, the antenna/coil of the relay component 12, the mouthpiece component 10, the mouthpiece coil(s) 11, or any combination thereof using NFMI (digital and/or analog), or vice versa. The earpiece components 100 can provide passive and/or active noise protection (also referred to as hearing protection). Passive noise protection is sound reduction that does not use a power source. Active noise protection is sound reduction that uses a power source. The earpiece components 100 can be made of one or more passive hearing protection (e.g., noise control) materials, for example, sound-damping material, sound-absorbing material, sound-reflecting material, sound-diffusing material, or any combination thereof. There are also physical shapes which when exposed to high audio wave forces clamp shut and attenuate the sound, but otherwise are open. The earpiece components 100 can have one or multiple passive and/or active filters. Passive filters can have one or more passive circuit components (e.g., resistors, capacitors, inductors) or can have no circuit components. Active filters can have one or more active circuit components (e.g., op amps). For example, the earpiece components 100 can have one or multiple mechanical filters, electronic filters, digital filters, or any combination thereof. Such filters can provide passive and/or active noise protection. For example, the mechanical filters can provide passive hearing protection, the digital filters can provide active hearing protection, and the electronic filters can provide passive and/or active hearing protection.

The earpiece components 100 can actively and/or passively dampen (also referred to as attenuate) sound, for example, environmental noise 102. The earpiece components 100 can actively and/or passively dampen electronic sound signals, for example, audio signals received from the mouthpiece component 10 and/or the relay component 12. The earpiece components 100 can be configured to dampen one or multiple sound wave frequencies. The earpiece components 100 can be configured to dampen one or multiple sound wave frequency bands (also referred to as ranges). The earpiece components 100 can be configured to dampen any sound wave frequency and/or frequency range within and/or outside the limits of human hearing, as different users may have different sensitivities to sound, different environments may have widely varying noise conditions and/or impulse events, and sounds outside a user's perception may still be at harmful pressures that should be attenuated. For example, the earpiece components 100 can be configured to dampen any sound wave frequency and/or frequency range within or bounded by the following spectrums: from about 0 Hz to about 40,000 Hz, more narrowly from about 0 Hz to about 35,000 Hz, yet more narrowly from about 0 Hz to about 20,000 Hz, including, for example, every 1 Hz frequency increment within these spectral ranges, and every frequency band within these spectral ranges of about 10 Hz, about 100 Hz, about 1,000 Hz, about 5,000 Hz, about 10,000 Hz, about 15,000 Hz, or any combination thereof.

The earpiece components 100 can be configured to dampen sound waves independent of frequency and/or can be configured to dampen sound waves dependent on frequency. The earpiece components 100 can be configured to dampen sound waves uniformly or non-uniformly across a sound's spectral composition (also referred to as its frequency domain).

The earpiece components 100 can have one or more low-pass filters, high-pass filters, band-pass filters, band-reject filters, notch filters, comb filters, all-pass filters, impulse noise filters, equalizers, compressors, or any combination thereof to achieve the desired damping profile and/or damping effect. For example, the earpiece components 100 can be configured to dampen soft noises and/or loud noises. Soft noises can be dampened the same or a different amount than loud noises. For example, soft noises can be dampened less than, the same as, or more than loud noises. Similarly, loud noises can be dampened less than, the same as, or more than soft noises. The earpiece components 100 can provide impulse noise protection, for example, with one or more impulse noise filters. The one or more filters used for damping can be passive and/or active filters.

The system can use higher-than-human-hearing frequencies to send data (such as electromagnetic control data signals which are beyond the range of human perception) to the earpieces 100. Such signals can be sent using analog NFMI (e.g., from the relay component 12), and is a way for analog base-band NFMI earpieces 100 to receive control signals from the relay component 12. A filter inside the earpiece 100 can isolate the signals at the high audio frequency (i.e., at the frequency or frequencies at higher-than-human-hearing levels). The system can use such isolated signals as control signals. For example, a 25 kHz tone can be sent along with the audio, which when present can tell the earpiece 100 to reduce the volume of external sounds to allow the user to concentrate on the incoming audio communications. The relay component 12 can send the 25 kHz tone and the audio.

An alternative way that any of the components may communicate with one another on the body may include the transmission of audio signals above the frequency range of human perception. A short high-frequency signal such as a pre-“chirp” could be sent immediately prior to the audio being transmitted to the mouthpiece, and this pre-“chirp” could be used as a data signal to indicate to the earpiece components to attenuate environmental sound.

The earpiece components 100 can analyze an incoming spectral audio band to determine which frequencies to attenuate. The earpiece components 100 can attenuate frequencies that have an amplitude or average amplitude that is greater than or equal to an amplitude threshold. The earpiece components 100 can determine which frequencies are loud and which frequencies are soft. Soft noises can have a first amplitude or first average amplitude less than a first amplitude threshold and loud noises can have a second amplitude or second average amplitude greater than or equal to a second amplitude threshold. The first and second amplitude thresholds can be the same or different from one another.

The earpiece components 100 can be configured to amplify one or more sounds and/or electronic sound signals by amplifying one or more sound frequencies and/or frequency ranges. For example, the earpiece components 100 can be configured to amplify soft noises and/or loud noises. Soft noises can be amplified the same or a different amount than loud noises. For example, soft noises can be amplified less than, the same as, or more than loud noises. Similarly, loud noises can be amplified less than, the same as, or more than soft noises.

The earpiece components 100 can provide dynamic range compression, for example, with one or more compressors. The one or more compressors can be configured to reduce the level (also referred to as volume) of sound or an audio signal that has an amplitude that exceeds a downward compression threshold and/or can be configured to increase the level (also referred to as volume) of sound or an audio signal that has an amplitude that falls below an upward compression threshold. The downward compression threshold can be about −100 dB to about 200 dB, for example, about −50 dB. The upward compression threshold can be about −100 dB to about 200 dB, for example, 110 dB. The downward compression threshold can be the same or different from the upward compression threshold. For example, the downward and upward compression thresholds can be about −50 dB and about 50 dB, respectively. As another example, the downward and upward compression thresholds can be about −110 dB and about 110 dB, respectively.

The earpiece components 100 can have one or multiple attenuation level settings. The attenuation level settings can comprise one or multiple discreet levels or a continuously adjustable level across an attenuation level spectrum. For example, the earpiece components 100 can have 1-5 attenuation levels or gain levels to amplify received sound (e.g., 1, 2, 3, 4, or 5 attenuation levels), or can range continuously from a minimum level to a maximum level across an attenuation level spectrum. The attenuation level spectrum and/or attenuation levels can be static or can be automatically adjusted based at least partly on the sound and/or sound signals received by the earpiece components 100. The one or multiple attenuation level settings can correspond to one or multiple impulse protection levels. The impulse protection level(s) can remain at the level for safe hearing regardless of any volume and/or attenuation settings of any component of the system.

The earpiece components 100 can have one or more controls (e.g., one or more multi-state controls) that can be manually or automatically manipulated to adjust the attenuation level, for example, from a first attenuation level setting to a second attenuation level setting. The controls can be one or more manipulatable mechanisms, for example, buttons, switches, knobs, or any combination thereof. The manipulatable mechanisms can be translatable and/or rotatable. The controls can be one or more touch screens or touch surfaces. The controls can be one or more voice controls (e.g., voice control interfaces) that can receive user commands (e.g., “increase attenuation,” “decrease attenuation,” “amplify frequency,” “dampen frequency,” “turn on,” “turn off”). The earpiece components 100 can receive a voice command, for example, from the mouthpiece components 10, from the relay component 12, and/or from the earpiece components 100 (e.g., as an environmental sound). A user can adjust the attenuation setting between the different levels and/or continuously across the attenuation level spectrum, for example, by manually adjusting/using one or more of the controls, touch screen/surface controls, voice controls, or any combination thereof. Additionally or alternatively, one or more of the controls (e.g., all of the controls) can be dictated over a wireless link to the relay component 12.

The earpiece components 100 can be configured to automatically adjust the attenuation setting between the different levels and/or continuously across the attenuation level spectrum based at least partly on the environmental noise and/or sound signals received. The earpiece components 100 can be configured to dynamically adjust the attenuation of environmental sounds when provided one or more status signals (e.g., to increase attenuation when incoming communications are occurring to either the earpiece component 100 or the mouthpiece component10). The earpiece components 100 can be configured to sense a status signal and thereafter dynamically adjust the attenuation of the environmental sounds being received. The status signal can be a wireless status signal. The status signal can be, for example, a power signal (e.g., a digital signal decoded from a wireless link) from the earpiece components 100 and/or a signal that corresponds to when the wireless earpiece components are connected to the system.

The earpiece components 100 can have one or multiple gain level settings. The gain level settings can comprise one or multiple discreet levels or a continuously adjustable level across a gain level spectrum. For example, the earpiece components 100 can have 1-5 gain levels (e.g., 1, 2, 3, 4, or 5 gain levels), or can range continuously from a minimum level to a maximum level across the gain level spectrum. The gain level spectrum and/or gain levels can be static or can be automatically adjusted based at least partly on the sound and/or sound signals received by the earpiece components 100.

The earpiece components 100 can have one or more controls (e.g., one or more multi-state controls) that can be manually or automatically manipulated to adjust the gain level, for example, from a first gain level setting to a second gain level setting. The controls can be one or more manipulatable mechanisms, for example, buttons, switches, knobs, or any combination thereof. The manipulatable mechanisms can be translatable and/or rotatable. The controls can be one or more touch screens or touch surfaces. The controls can be one or more voice controls (e.g., voice control interfaces) that can receive user commands (e.g., “increase gain,” “decrease gain,” “amplify frequency,” “dampen frequency,” “turn on,” “turn off”). The earpiece components 100 can receive a voice command, for example, from the relay component 12 and/or from the earpiece components 100 (e.g., as an environmental sound). A user can adjust the gain setting between the different levels and/or continuously across the gain level spectrum, for example, by manually adjusting/using one or more of the controls, touch screen/surface controls, voice controls, or any combination thereof.

A user can manually adjust the gain setting between the different levels and/or continuously across the gain level spectrum. The earpiece components 100 can automatically adjust the gain setting between the different levels and/or continuously across the gain level spectrum based at least partly on the environmental noise received.

The earpiece components 100 can be configured to automatically adjust the gain setting between the different levels and/or continuously across the gain level spectrum where the gain and attenuation level range are a continuum of the same parameter based at least partly on the environmental noise and/or sound signals received. The earpiece components 100 can be configured to dynamically adjust the gain of environmental sounds when provided with one or more status signals (e.g., to decrease gain when a noise impulse event or a high external noise level is detected). The earpiece components 100 can be configured to sense a status signal and thereafter dynamically adjust the gain of the environmental sounds being received. The status signal can be a wireless status signal.

The earpiece components 100 can have one or multiple volume level options for incoming communications (e.g., as volume level can be different than the gain setting). The earpiece components 100 can have a continuously adjustable volume level that can range from a minimum volume (e.g., no sound) to a maximum volume for incoming communications. The incoming communications can be environmental noise and/or sound signals received from the mouthpiece component 10 and/or the relay component 12. FIG. 4 further illustrates that the earpiece component 100 can have one or more volume controls 112. For example, the earpiece components 100 can have a first volume control 112a to adjust the volume of external sound 102 and a second volume control 112b to adjust the volume of the sound received from the relay component 12, the antenna/coil of the relay component 12, the mouthpiece component 10, the mouthpiece coil(s) 11, or any combination thereof.

The volume controls 112 can be one or more manipulatable mechanisms, for example, buttons, switches, knobs, or any combination thereof. The manipulatable mechanisms can be translatable and/or rotatable. The volume controls 112 can be one or more touch screens or touch surfaces. The volume controls 112 can be one or more voice controls (e.g., voice control interfaces) that can receive user commands (e.g., “increase volume,” “decrease volume,” “amplify frequency,” “dampen frequency,” “turn on,” “turn off”). A user can adjust the volume between the different volume levels and/or continuously from a minimum volume (e.g., no sound) to a maximum volume, for example, by manually adjusting/using one or more of the controls, touch screen/surface controls, voice controls, or any combination thereof.

The one or more controls 112 can control the attenuation levels, gain levels, volume levels, or any combination thereof. The one or more controls 112 are also referred to as a control interface 112, as the controls 112 can take myriad forms. The controls/control interfaces 112 can receive the system control signals 16. The system controls 16 can include any controllable parameter of the system, for example, the attenuation levels, gain levels, volume levels, or any combination thereof.

The earpiece components 100 can provide passive noise isolation by physically interfering with (also referred to as blocking) environmental noise, for example, by absorbing and/or reflecting environmental noise.

The earpiece components 100 can provide active noise cancellation by electronically canceling environmental noise. For example, the earpiece components 100 can be configured to analyze incoming sound and emit a reverse-phase audio that cancels the incoming sound via destructive interference.

The earpiece components 100 can improve a user's ability to communicate in, for example, high noise environments given that the earpiece components 100 can protect a user's hearing, dampen sound, pass-through sound, amplify sound, or any combination thereof as described above.

FIG. 4 further illustrates that a user can wear the earpiece components 100 in addition to or in lieu of the mouthpiece component 10. The simpler configurations of the communication system involve either no earpiece component 100 (open-ears) or various forms of passive or active hearing protection without integrated communications (e.g., the passive earpiece components 100 described herein). The open-ears option is illustrated in FIG. 1 and has the advantage of non-visibility to an outside observer.

The hearing protection options all increase the visibility of the system, but have the advantage of reducing the distraction of competing audio inputs to the brain. By damping environmental noise, the user is able to more easily focus on the incoming audio through the teeth (via the mouthpiece 10), while simultaneously protecting their own hearing from loud environments. Clear communications are therefore possible in two directions, even in very loud environments, with minimal weight on the head (e.g., as compared to over-ear cups).

Once incoming communications are incorporated into the ear-worn components 100 as shown in FIG. 4, the benefits of the system are even greater (e.g., as compared to systems with earpiece components 100 without integrated communications). The hearing-protection benefits still exist, as outlined above, but in addition, the audio volume provided by the ear-component is able to exceed the maximum perceived audio level achieved by applying vibrations to the teeth at a comfortable level. This allows for increased SNR for received audio communications when in an extremely loud environment. Another desirable feature of systems with ear-worn components 100 with integrated communications is that they can be capable of full-duplex operation (e.g., in addition to half-duplex operation), where the user can listen to incoming audio while transmitting simultaneously. The fact that audio would be going to the ears and the speech coming from the mouth means there is essentially no feedback issue to contend with, which is a technical obstacle that must be overcome to achieve full-duplex operation with just a mouthpiece based audio interface.

An additional benefit comes from switching from wired to wireless ear-worn components 100 with active received audio capability. Reduction in wires and cable management needs is a benefit as well. To allow for this, the ear-worn components 100 can implement a wireless method of audio communications, and the relay component 12 of the system can support it. This may be done using various wireless protocols such as BT, BLE, or custom ISM band, but most simply by using analog NFMI and a base-band audio signal sent over the magnetic field.

Using ear-worn components 100 that integrate communication provide various system benefits. For example, using ear-worn components 100 with integrated communication gives the user and/or system the ability to dynamically optimize the balance between situational awareness and communications. Outside sounds can be passed through or even enhanced, and then attenuated when communications are incoming. This is beneficial regardless of whether the incoming audio is through the ears or teeth, so as to reduce environmental distraction. As another example, the earpiece components 100 can give users the ability to quickly transition from a low-visibility communications setup to a kinetic/tactical operation by simply donning the ear-component 100 of the system. That act simultaneously provides needed hearing protection during a kinetic operation, as well as enabling the additional incoming audio pathway with higher volume capabilities on receive audio to help overcome loud environmental noise that comes along with the kinetic operation. As yet another example, the earpiece components 100 enable full-duplex audio communications (e.g., in addition to half-duplex communications), allowing receipt of audio while the user is transmitting simultaneously.

FIG. 5 illustrates the system of FIG. 4, further allowing the relay component 12, the mouthpiece component 20, the wireless remote 109, and the earpiece component 100 to interface with one another via body conduction 27, such as utilizing eHBC or mHBC. Wireless communications between the mouthpiece component 10, the earpiece component 100, the relay component 12, and the infrastructure communication device 14 as shown in FIG. 5 should be selected for the particular operating environment (e.g. proximity to the body, in the body, in water), data rate/bandwidth, power requirements, detectability to an observer, susceptibility to external interference (e.g. coexistence with other devices or intentional jamming), and compatibility with infrastructure communications device and support for multiple channels.

An alternative embodiment can use WBAN (e.g. Bluetooth) between one of the earpieces and the infrastructure device (bypassing the relay component 12). The two earpiece components 100a, 100b can then communicate over a separate independent NFMI link or time or frequency shared NFMI link, with latency between ears handled in the first earpiece component 100a.

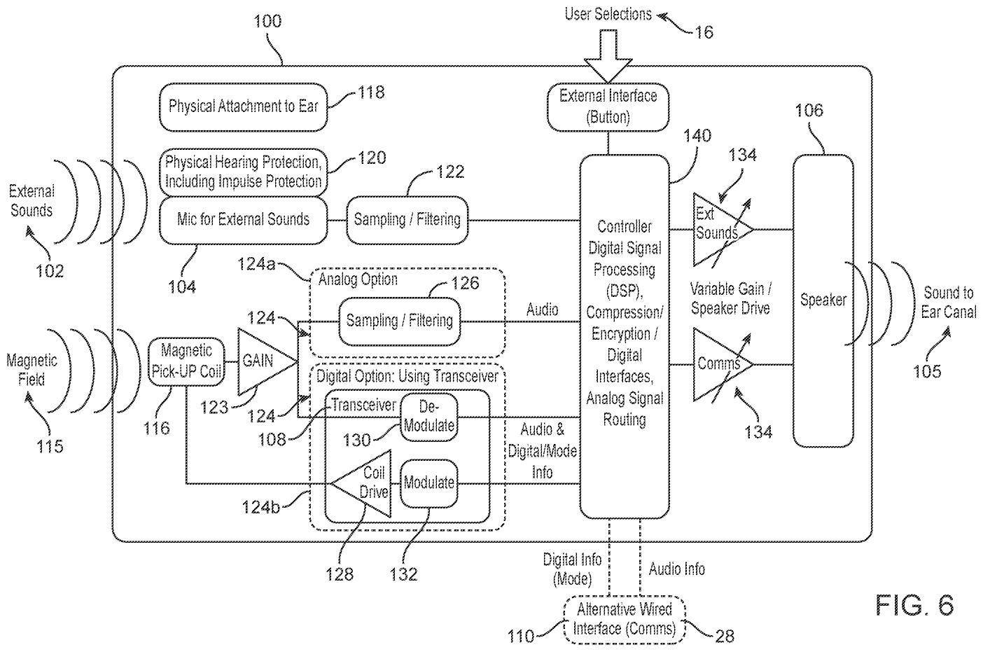

FIG. 6 illustrates a schematic variation of an earpiece component 100 capable of intra-system communication. The earpiece component 100 can also be capable of two-way communication. FIG. 6 illustrates that each earpiece component 100 can have one or more earpiece microphones 104, one or more earpiece speakers 106, one or more earpiece wireless components 116, or any combination thereof, for example, in the arrangement shown. The earpiece microphones 104 can receive external or internal sounds 102. The earpiece speakers 106 can emit sounds 105, for example, into an ear canal. The earpiece wireless components 116 can receive signals 115 from the relay component 12, the antenna/coil of the relay component 12, the mouthpiece component 10, the coil(s) 11 of the mouthpiece component 10, or any combination thereof. The signals 115 can be in the form of a magnetic field and be communicated to the earpiece wireless component 116, for example, via the antenna/coil of the relay component 12 and/or the coil(s) 11 of the mouthpiece component 10. Each of the earpiece microphone 104, the earpiece speaker 106, and/or the earpiece wireless component 116 can be attached to or integrated with the earpiece components 110, for example, attached to, positioned in, or integrated within an earpiece housing (not shown).

Each earpiece wireless component 116 can be one or more antennas and/or coils. The earpiece antennas/coils 116 can be magnetic pick-up antennas/coils. For example, the earpiece antennas/coils 116 can be NFMI antennas/coils 116 (e.g., a wire-wound ferrite core antenna and/or a wide loop option such as a loop coil). The earpiece antennas/coils 116 can enable the data links 28, 110 described above. The earpiece antennas/coils 116 can be attached to and/or integrated with the earpiece components 100. Alternatively, each earpiece wireless component 116 may also use one or more antennas for use with RF, e.g., BLUETOOTH®, ISM, etc. for receiving corresponding electromagnetic signals 115.

FIG. 6 further illustrates that the earpiece components 100 can be physically attached 118 to one or both ears. FIG. 6 further illustrates that the earpiece components 100 can provide hearing protection 120 as described above, for example, physical hearing protection such as impulse protection. The hearing protection 120 provided can be passive and/or active as described above.

FIG. 6 further illustrates that the sound received by the one or more microphones 104 can be sampled and/or filtered 122 before being communicated/sent to a controller 140.

FIG. 6 further illustrates that the signals 115 received by the one or more wireless components 116 can be processed 124 before being sent to the controller 140. For example, the audio signals 115 can undergo analog processing 124a and/or digital processing 124b. FIG. 6 further illustrates that be audio signals 115 can be amplified 123 prior to being processed 124 and sent to the controller 140.

The analog processing 124a can include one or more sampling and/or filtering components 126. The digital processing 124b can include one or more earpiece transceivers 108, one or more coil drives 128, one or more de-modulators 130, one or more modulators 132, or any combination thereof. The signal output from the processing 124 can be sent to the controller 140. The controller 140 can perform digital signal processing (DSP), compression, encryption, digital interfaces, analog signal routing, or any combination thereof on the various audio signals received. FIG. 6 further illustrates that the controller 140 can be configured to receive audio signals from the earpiece microphones 104, from the wireless components 116, from the controls/control interfaces 112, or any combination thereof.

The controller 140 can send output signals to the earpiece speaker 106. One or more variable gain and/or speaker drives 134 can process the controller output signals. The earpiece speaker 106 can convert the controller output signals to sound 105.

A user can select which of the one or multiple independent receivable audio pathways to receive (and listen to). For example, a control/control interface (e.g., one or more controls/control interfaces 112) can enable a user to select between the mouthpiece component 10 and one or both of the earpiece components 100.

The system can automatically select which of the one or multiple independent receivable audio pathways a user receives (and can listen to), for example, based on one or more of the status signals described above.

The system can enable a user to receive an audio pathway alone or separate from another audio pathway. For example, when the system has multiple audio pathways, the system can activate one audio pathway at a time. The activated audio pathway can be routed to the appropriate sound or vibration emission source, for example, the one or more speakers 106 of a left earpiece component 100a, the one or more speakers 106 of a right earpiece component 100b, or the one or more transducers 18 of the mouthpiece component.

The system can enable a user to receive multiple audio pathways simultaneously. For example, when the system has multiple audio pathways, the system can activate multiple audio pathways at a time (e.g., 2, 3, 4, 5, 6, 7, or more audio pathways). The activated audio pathways can be routed to the appropriate sound or vibration emission source, for example, the one or more speakers 106 of a left earpiece component 100a, the one or more speakers 106 of a right earpiece component 100b, the one or more transducers 18 of the mouthpiece component, or any combination thereof.

Regardless of the incoming audio path chosen, the mouthpiece component 10 may be used as the microphone, which enjoys extremely good voice SNR due to physical placement, intentional attenuation, and body noise shielding.

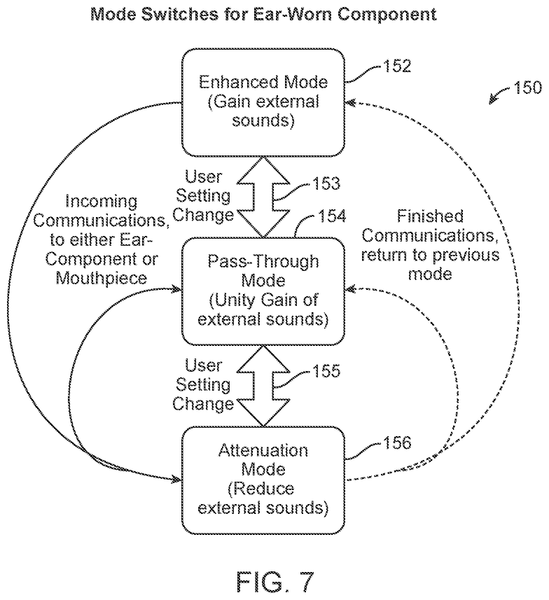

FIG. 7 is a schematic illustration of a variation of various audio processing modes 150 of an earpiece component 100 for incoming communications. The various audio modes 150 can each process external sound 102 received by the earpiece component 100 differently from one another. For example, the system can have an enhanced mode 152 that can gain external sounds 102, a pass-through mode 154 that can perform or have a unity gain of external sounds 102, an attenuation mode 156 that can dampen external sounds 102, or any combination thereof. Transitions between and among the different modes 152, 154, and/or 156 can occur automatically when incoming communications arrive. The system can automatically apply the different modes 152, 154, and/or 156 as needed, dependent on the incoming communications received. For example, when the relay component 12 receives communications from the mouthpiece component 10 and/or when the earpiece component 100 receives communications from the relay component 12, the system can automatically apply mode 152, mode 154, or mode 156 as needed without any input from the user.

Additionally or alternatively, the various modes 150 (e.g., mode 152, mode 154, and/or mode 156) can be selectable by the user in real-time. For example, the different modes 150 can each be selectable using one or more audio controls. Each audio mode 150 can be associated with a separate audio control, for example, a first audio mode (e.g., enhanced mode 152) can be associated with a first audio control, a second audio mode (e.g., pass-through mode 154) can be associated with a second audio control, and a third audio mode (e.g., attenuation mode 156) can be associated with a third audio control. The first, second and/or third controls can be selectable (e.g., separately, sequentially and/or simultaneously selectable) by a user to turn on, turn off, and/or adjust (e.g., increase or decrease the effect of the audio mode) the audio modes 150 associated with each control. Each audio mode 150 can be associated with a separate setting of a single audio control, for example, a first audio mode (e.g., enhanced mode 152) can be associated with a first setting of an audio control, a second audio mode (e.g., pass-through mode 154) can be associated with a second setting of the audio control, and a third audio mode (e.g., attenuation mode 156) can be associated with a third setting of the audio control. The first, second and/or third settings can be selectable (e.g., separately, sequentially and/or simultaneously selectable) by a user to turn on, turn off, and/or adjust (e.g., increase or decrease the effect of the audio mode) the audio modes 150 associated with each control setting. Multiple audio modes 150 can be associated with one or more single audio controls. For example, a first, second, and/or third audio mode (e.g., modes 152, 154, and/or 156) can be associated with a first control and/or control setting. Additionally, or in combination, the first, second and/or third audio mode (e.g., modes 152, 154, 156) can be associated with a second control and/or control setting. For example, the first control and/or control setting can be associated with low frequency sound and the second control and/or control setting can be associated with high frequency sound. As an example, two or three of the different modes 150 can be controlled with a single controller. One or more of the audio controls and/or control settings can be dependent on one or more of the other audio controls and/or control settings. For example, in one variation, only one mode 150 can be activated at a time. This can be used, for example, where the different modes 150 each filter the same frequencies or frequency bands. In another variation, two or more modes can be simultaneously activated. For example, different modes can be simultaneously activated where the different modes filter different frequencies or different frequency bands.

The audio controls can be switches, buttons, knobs, touch screens, touch surfaces, any combination thereof, or any other selectable interface. For example, the different modes 150 can be controlled with the one or multiple controls/control interfaces 112 shown in FIG. 4. Each audio control (e.g., control 112) can have one or more control settings as described above, for example, fully off, fully on, or partially on/off, where “off” corresponds to an inactivated state and “on” corresponds to an activated state. The partially on setting can correspond to any percentage activation between 0% and 100%, for example, 25% activation, 50% activation, 75% activation, or any other percentage between 0% and 100%. The modes 150 can correspond to three settings of a control/control interface 112 (e.g., three settings/positions of a switch, button, knob, touch screen, touch surface, etc.). The modes 150 can correspond to settings (e.g., on/off settings) of three different controls/control interfaces 112.

FIG. 7 further illustrates that a user can change from the enhanced mode 152 to the pass-through mode 154 via a user input 153 (and vice versa), and that a user can change from the pass-through mode 154 to the attenuation mode 156 via a user input 155 (and vice versa). Alternatively or additionally, FIG. 7 further illustrates that the system can automatically change from the enhanced mode 152 to the pass-through mode 154 without the user input 153 (and vice versa), and that the system can automatically change from the pass-through mode 154 to the attenuation mode 156 without the user input 155 (and vice versa). When the enhanced or attenuation mode 152, 154 is selected (e.g., by the system or the user), the gain or attenuation of external sounds 102 can be adjusted as described above, for example, automatically or with the same or a different control/control interface 112 that was used to select between the different modes 150. FIG. 7 further provides that incoming communications can be routed to the earpiece components 100 and/or the mouthpiece component 10, and that the finished communications can return to the previous mode. For example, the incoming communications can be routed to the earpiece components 100 independently of the external sound attenuation mode selected (e.g., by the user) or activated (e.g., by the system), and can optionally help to automatically select the attenuation mode as shown by the arrows in FIG. 7. The communications come in (e.g., to the mouth and/or the ears), and the external sound attenuation can be increased during that time, and return to the previous settings afterwards.

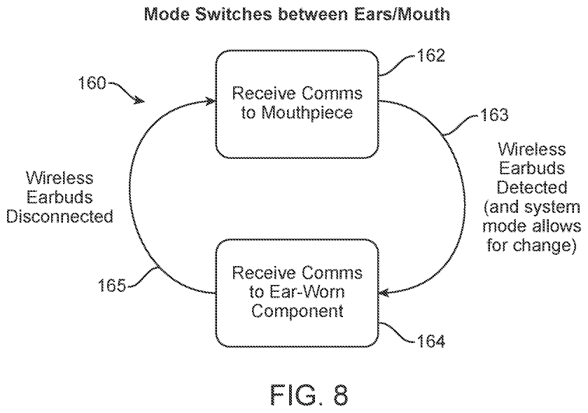

FIG. 8 is a schematic illustration of a variation of audio reception modes 160 of the system for incoming communications, for example, the system can have a mouthpiece component reception mode 162 and/or an earpiece component reception mode 164. FIG. 8 further illustrates that the incoming audio communications can be routed to the ears and/or mouth as desired with one or more of the audio reception modes 160 (e.g., modes 162 and 164). The system can have, for example, two incoming audio reception modes 162, 164 so that the earpiece components 100 (e.g., via mode 164) and/or the mouthpiece component 10 (e.g., via mode 162) can be manually or automatically selected to communicate sound (e.g., sound waves from an earpiece microphone 106) or sound signals (e.g., vibrations from a mouthpiece transducer 18) to a user. When the mouthpiece component reception mode 162 is activated, for example, by being manually or automatically selected, the user can receive communications from the mouthpiece component 10. When the earpiece component reception mode 164 is activated, for example, by being manually or automatically selected, the user can receive communications from the earpiece component 100. Another option may include sending audio signals to both the mouthpiece component 10 and earpiece components 100 simultaneously for increased perception of volume or at slightly different times to allow for three-dimensional audio with three points of insertion. The modes 160 (e.g., mode 162 and/or mode 164) can be manually selected by the user or automatically selected by the system. For example, the system can automatically select the mouthpiece component reception mode 162 when the left and/or right wireless earpiece components 100a, 100b are not connected to the system and/or are disconnected from the system, shown for example by arrow 165. As another example, the system can automatically select the earpiece component reception mode 164 when one or more earpiece components 100 are detected or otherwise connected to the system, shown for example by arrow 163. The system can be configured to switch from the mouthpiece component reception mode 162 to the earpiece component reception mode 164 if an earpiece component 100 is detected with or without additional input from the user and/or with or without another detected event (e.g., a malfunction of the mouthpiece component, at least two separate incoming sound sources are detected, etc.). The earpiece components 100 can be considered connected to the system when they are in wireless communication with another component of the system, for example, the mouthpiece component 10, the relay component 12, and/or the infrastructure communication device 14. The earpiece components 100 can be considered connected when engaged with a user's ear. Engagement with a user's ear can be detected, for example, with a pressure sensor attached to or integrated with the earpiece components 100.

The system can have one or more mode controls that the user can manually select, for example, one or more switches, buttons, knobs, touch screens, touch surfaces, any combination thereof, or any other selectable interface. The user can use such mode controls to select between one or more sources of incoming communication, for example, between communication from the mouthpiece component 10 and/or communication from the earpiece components 100. Where the system has two or more earpiece components 100, each earpiece component 100 can have its own mode control. For example, the left earpiece component 100a can have a first mode control and the right earpiece component 100b can have a second mode control different from the first mode control. Additionally, or in combination, where the system has two or more earpiece components 100, one of the earpiece components 100, the relay component 12, and/or the infrastructure communication device 14 can have a mode control that the user can selectively activate to control audio reception in the two or more earpiece components 100. Alternatively or additionally, the system can have an application running on a personal computer (PC) that can configure the system for the different audio routing options. The PC can have a graphical user interface (GUI) and can send the selections to the system over a wireless communication link. For example, the GUI can display various audio routing options that the user can select by interacting with the GUI (e.g., touching the screen). The PC can send the selection(s) to the system. The PC can be any computer, for example, a smartwatch, a smartphone, a tablet, or any other personal computing device.

Each earpiece component 100 can have the same or different noise processing and/or modifying properties, the same or different components, and/or the same or different communication protocols described herein relative to one or more other earpiece components 100, for example, the same or different processing properties and/or components, the same or different damping properties and/or components, the same or different amplifying properties and/or components, the same or different noise cancellation properties and/or components, the same or different communication protocols and/or components, or any combination thereof. The noise processing and/or modifying properties, components and communication protocols described herein can be combined in any combination in any one or more earpiece components 100, as every permutation of disclosed features (e.g., system properties and components) and processes (e.g., communication protocols) is hereby explicitly disclosed.

Each of these implementations may include capabilities for transfer of data other than audio data between the head and the infrastructure devices 14. This may include biometric sensors in the mouthpiece component 20 or earpiece components 100 that monitor signals related to health or environment of the user, e.g. heartbeat, temperature, respiration, hydration, blood oxygenation, movement, orientation, acceleration/shock, location. This data can be transferred wirelessly in parallel with the audio stream or time interleaved with the audio stream for storage at the relay component 12 or transmitted over the infrastructure devices 14.

Further, the system may include means (e.g. polling of slaves by the client) to identify presence of all wireless components (e.g. the mouthpiece component 20, the earpiece components 100, the wireless remote 109, the relay component 12) and alert the user via visual notification (e.g. LED on the relay component 12) or audio notification to the mouthpiece component 20 or earpiece components 100.

All references, including patent applications and publications, cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art.

The specific embodiments described herein are offered by way of example only. Moreover, such devices and methods may be applied to other sites within the body. Modification of the above-described assemblies and methods for carrying out the invention, combinations between different variations as practicable, and variations of aspects of the invention that are obvious to those of skill in the art are intended to be within the scope of the claims.

What is claimed is:

1. A two-way communication system, comprising:
a mouthpiece component removably engageable within a mouth of a user;
a relay component in wireless communication with the mouthpiece component via a wireless body area networking signal or a low-frequency non-propagating inductive field; and
an infrastructure communication device configured to transmit wireless signals to and receive wireless signals from the relay component, wherein the relay component is configured to interface between the mouthpiece component and the infrastructure communication device.

2. The two-way communication system of claim 1, wherein the relay component is in wireless communication with the mouthpiece component via a magnetic resonant coupling.

3. The two-way communication system of claim 2,
wherein the mouthpiece component comprises a first coil configured to produce a first magnetic field through the user's body,
wherein the relay component comprises a second coil configured to produce a second magnetic field through the user's body, and
wherein the first coil is configured to resonantly couple with the second magnetic field from the second coil and the second coil is configured to resonantly couple with the first magnetic field from first coil when propagated through tissue of the user's body for wireless communication.

4. The two-way communication system of claim 1, further comprising a first earpiece component positionable with or in proximity to an ear of the user, wherein the first earpiece is in wireless communication with the mouthpiece component and the relay component.

5. The two-way communication system of claim 4, wherein the first earpiece is in wireless communication with the mouthpiece component and the relay component via magnetic resonant coupling.

6. The two-way communication system of claim 5,
wherein the first earpiece component comprises a third coil configured to produce a third magnetic field through the user's body, and
wherein the third coil is configured to resonantly couple with the first and second magnetic field respectively from the first and second coils and the first and second coils are configured to resonantly couple with the third magnetic field from third coil when propagated through tissue of the user's body for wireless communication.

7. The two-way communication system of claim 6, further comprising
a second earpiece component positionable with or in proximity to another ear of the user, the second earpiece component comprising a fourth coil configured to produce a fourth magnetic field through the user's body,
wherein the fourth coil is configured to resonantly couple with the third magnetic field from the third coil and the third coil is configured to resonantly couple with the fourth magnetic field from fourth coil when propagated through tissue of the user's body for wireless communication.

8. The two-way communication system of claim 7, wherein the first, second, third, or fourth coil comprises a transmitting coil configured to produce the first, second, third, or fourth magnetic field, respectively, through the user's body, and wherein the first, second, third, or fourth receiving coil is configured to resonantly couple with one of the other magnetic fields.

9. The two-way communication system of claim 1, wherein the relay component is integrated with the infrastructure communication device.

10. The two-way communication system of claim 1, wherein the relay component is in wireless communication with the mouthpiece component via the wireless body area networking signal or the low-frequency non-propagating inductive field comprising a MICS or ISM link.

11. The two-way communication system of claim 1, wherein the relay component is in wireless communication with the mouthpiece component via the wireless body area networking signal low-frequency non-propagating inductive field comprising a low or high UWB link.

12. The two-way communication system of claim 1, wherein the relay component is in wireless communication with the mouthpiece component via the wireless body area networking signal low-frequency non-propagating inductive field comprising a galvanic or capacitive HBC link.

13. The two-way communication system of claim 1, wherein the relay component is in wireless communication with the mouthpiece component via the wireless body area networking signal low-frequency non-propagating inductive field comprising a wireless magnetic NFMI or HBC link.

14. The two-way communication system of claim 1, wherein the infrastructure communication device transmits and receives wireless signals relative to the relay component via a MICS or ISM link.

15. The two-way communication system of claim 1, wherein the infrastructure communication device transmits and receives wireless signals relative to the relay component via a low or high UWB link.

16. The two-way communication system of claim 1, wherein the infrastructure communication device transmits and receives wireless signals relative to the relay component via a galvanic or capacitive HBC link.

17. The two-way communication system of claim 1, wherein the infrastructure communication device transmits and receives wireless signals relative to the relay component via a wireless magnetic NFMI or HBC link.

18. The two-way communication system of claim 1, wherein the relay component and the mouthpiece component are further communicable over a MICS link with a frequency band of 402 MHz to 405 MHz.

19. The two-way communication system of claim 1, wherein the relay component and the mouthpiece component are further communicable over an ISM link with a frequency band of 433 MHz to 434.8 MHz.

20. The two-way communication system of claim 1, wherein the low-frequency non-propagating inductive field comprises a NFMI link with a frequency band less than 15 MHz.

21. The two-way communication system of claim 1, wherein the relay component and the mouthpiece component are further communicable over galvanic or capacitive coupling.

22. The two-way communication system of claim 1, wherein the relay component further comprises a push-to-talk component.

23. The two-way communication system of claim 1, wherein the mouthpiece component comprises a biometric sensor wherein biometric data is transferred wirelessly to the relay component.

24. The two-way communication system of claim 1, further comprising a wireless remote configured to wirelessly link to the relay component.

25. The two-way communication system of claim 4, wherein the first earpiece component is configured to receive a voice command from the mouthpiece component or the relay component.

26. The two-way communication system of claim 1, wherein sensors are incorporated on the mouthpiece component to allow for tongue actuation for system controls.

27. The two-way communication system of claim 1, wherein the relay component processes audio data to and from the mouthpiece component.

28. The two-way communication system of claim 1 wherein an infrastructure communication device is a tactical radio or smartphone.

29. A two-way communication system, comprising:
a mouthpiece component removably engageable within a mouth of a user;
a first earpiece component positionable with or in proximity to an ear of the user;
a relay component in in wireless communication with the mouthpiece component and/or first earpiece component via a wireless body area networking signal or a low-frequency non-propagating inductive field; and
an infrastructure communication device configured to transmit wireless signals to or receive wireless signals from the relay component,
wherein the relay component is configured to interface between the mouthpiece component and/or the first ear piece component and the infrastructure communication device.

30. The two-way communication system of claim 29, wherein the relay component is in wireless communication with the mouthpiece component and/or first earpiece component via a magnetic resonant coupling.

31. The two-way communication system of claim 30, wherein the mouthpiece component contains a first transmitting coil configured to produce a first magnetic field through the user's body and a first receiving coil, wherein the relay component comprising a second transmitting coil configured to produce a second magnetic field through the user's body and a second receiving coil, and wherein the first receiving coil is configured to resonantly couple with the second transmitting coil and the second receiving coil is configured to resonantly couple with the first transmitting coil propagating through tissue of the user's body for wireless communication.

32. The two-way communication system of claim 31, wherein the first earpiece component comprises a third transmitting coil configured to produce a third magnetic field through the user's body and a third receiving coil, wherein the third receiving coil is configured to resonantly couple with the first or second transmitting coil and the first and second receiving coils are configured to resonantly couple with the third transmitting coil for propagating through tissue of the user's body for data transfer.

33. The two-way communication system of claim 32, further comprising a second earpiece component positionable with or in proximity to another ear of the user, the second earpiece component comprising a fourth transmitting coil configured to produce a fourth magnetic field through the user's body and a fourth receiving coil, wherein the fourth receiving coil is configured to resonantly couple with the third transmitting coil and the third receiving coil is configured to resonantly couple with the fourth transmitting coil propagating through tissue of the user's body for wireless communication.

34. The two-way communication system of claim 31, wherein the first and/or second earpiece components comprise button actuation features.

35. The two-way communication system of claim 29, wherein the relay component is integrated with the infrastructure communication device.

36. The two-way communication system of claim 29, wherein the resonance frequency band of the low-frequency non-propagating inductive field is less than 15 MHz.

37. The two-way communication system of claim 29, wherein the mouthpiece component further comprises a mouthpiece microphone.

38. The two-way communication system of claim 37, wherein the mouthpiece microphone comprises a MEMS-type air microphone.

39. The two-way communication system of claim 37, further comprising an attenuation element in vibrational communication with the mouthpiece microphone.

40. The two-way communication system of claim 37, further comprising one or more transducers incorporated into the mouthpiece component and configured to be vibrationally coupled to the one or more teeth.

41. The two-way communication system of claim 37, wherein the relay component further comprises a push-to-talk component.

42. The two-way communication system of claim 29, wherein the mouthpiece component comprises a biometric sensor wherein biometric data is transferred wirelessly to the relay component.

43. The two-way communication system of claim 29, wherein the relay component and the infrastructure communication device are communicable over a NFMI link.

44. The two-way communication system of claim 29, further comprising a wireless remote configured to wirelessly link to the relay component.

45. The two-way communication system of claim 29, wherein the first earpiece component is configured to receive a voice command form the mouthpiece component or the relay component.

46. The two-way communication system of claim 29, wherein sensors are incorporated on the mouthpiece component to allow for tongue actuation for system controls.

47. The two-way communication system of claim 29, wherein the relay component processes audio data to and from the mouthpiece component.

48. The two-way communication system of claim 29, wherein the infrastructure communication device is a tactical radio or smartphone.

49. A method of providing two-way communication, comprising:
receiving a signal via an infrastructure communication device carried by a user and configured to transmit wireless signals to or receive wireless signals from a relay component;
transmitting the signal to the relay component which is configured to interface between a mouthpiece component and the infrastructure communication device;
further transmitting the signal to the mouthpiece component which is configured for temporary securement upon a tooth or teeth of the user, wherein the relay component is in wireless communication with the mouthpiece component via a wireless body area networking signal or a low-frequency non-propagating inductive field; and
vibrationally conducting the signal through the mouth of the user via the mouthpiece component.

50. The method of claim 49, wherein the two-way communication system further comprises a first earpiece component that is in wireless communication with the mouthpiece component and/or the relay component.

51. The method of claim 50, further comprising attenuating the signal received by the first earpiece component via the infrastructure communication device if the signal exceeds a threshold level, wherein the infrastructure communication device is in wireless communication with the relay component and/or the first earpiece component.

52. The method of claim 51, further comprising:
switching reception of the signal to the mouthpiece component via the infrastructure communication device if the signal exceeds a threshold level, wherein the infrastructure communication device is in communication with the relay component and/or earpiece component; and
vibrationally conducting the signal through the one or more teeth of the user via the mouthpiece component.

53. A method of providing two-way communication, comprising:
receiving a signal from a mouth of a user via a mouthpiece component which is configured for temporary securement upon a tooth or teeth of the user;
transmitting the signal from the mouthpiece component to a relay component which is in wireless communication with the mouthpiece component via a wireless body area networking signal or a low-frequency non-propagating inductive field;
further transmitting the signal from the relay component to an infrastructure communication device carried by the user, wherein the relay component is configured to interface between the mouthpiece component and the infrastructure communication device; and
further transmitting the signal from the infrastructure communication device to a remote receiver, wherein the infrastructure communication device is configured to transmit wireless signals to or receive wireless signals from the relay component.

54. The method of claim 53, wherein the two-way communication system further comprises a first earpiece component that is in wireless communication with the mouthpiece component and/or the relay component.

55. The method of claim 54, further comprising attenuating the signal received by the first earpiece component via the infrastructure communication device if the signal exceeds a threshold level, wherein the infrastructure communication device is in wireless communication with the relay component and/or the first earpiece component.

56. The method of claim 55, further comprising switching reception of the signal to the mouthpiece component via the infrastructure communication device if the signal exceeds a threshold level, wherein the infrastructure communication device is in communication with the relay component and/or earpiece component.
